(19) United States
(12) Patent Application Publication
(10) Pub. No.: US 2026/0079162 A1
(43) Pub. Date: Mar. 19, 2026

(54) SYSTEMS, METHODS, AND KITS FOR DETECTING PROTEIN INTERACTIONS

(71) Applicant: Advanced Cell Diagnostics, Inc., Newark, CA (US)

(72) Inventors: Li-Chong Wang, Newark, CA (US); Maithreyan Srinivasan, Newark, CA (US); Ge-Ah Kim, Newark, CA (US); Sonali Anantprakash Deshpande, Newark, CA (US); Vasudha Murlidhar, Newark, CA (US); Emerald Doolittle, Newark, CA (US)

(21) Appl. No.: 19/132,600

(22) PCT Filed: Nov. 22, 2023
(86) PCT No.: PCT/US2023/080939

§ 371 Date: May 23, 2025

Related U.S. Application Data
(60) Provisional application No. 63/384,815, filed on Nov. 23, 2022.

(51) Int. Cl. G01N 33/68; G01N 33/53; G01N 33/542

(52) U.S. Cl. CPC G01N 33/6845; G01N 33/5308; G01N 33/542

(57) ABSTRACT

Disclosed herein are methods for detecting target protein interactions in a biological sample. Kits for carrying out the methods are also provided.

### CROSS-REFERENCE TO RELATED APPLICATIONS

This application claims the benefit of U.S. Provisional Application No. 63/384,815, filed Nov. 23, 2022, the content of which is herein incorporated by reference in its entirety.

### FIELD

Embodiments of the present disclosure include methods for detecting target protein interactions in a biological sample. Kits for carrying out the methods are also provided.

### BACKGROUND

Immunohistochemistry (IHC) and immunocytochemistry (ICC) are powerful techniques that are used to detect and localize specified proteins within tissue sections and cells, while maintaining spatial resolution and cytological context. IHC and ICC have broad and complementary applications in research and diagnostics. See Shi et al., Journal of Histochemistry & Cytochemistry 59(11): 13-32 (2011). For example, both approaches provide researchers insights regarding cell identities and states.

Complete characterization of complex cellular interactions within a tissue or cell requires a multi-omic strategy. For example, detecting and analyzing transcriptomic and proteomic information is informative in interrogating complex tissues and in revealing cell-type specific gene expression (see Vanlandewijck et al., Nature 554(7693): 475-482 (2018); Stempl et al., the Journal of Molecular Diagnostics 14(1): 22-29 (2014)), in identifying the cellular sources of secreted proteins (see Liou et al., Cell Reports 19(7): 1322-1333 (2017)), and in visualizing the spatial organization of the various cell types and their interactions. To fully and accurately characterize cells and tissues, protein interactions need to be detected in the same tissue sample, e.g., simultaneously, in a spatially resolved manner.

### SUMMARY

Provided herein are methods and kits for detecting protein interactions.

In one aspect, the methods comprise (i) contacting a biological sample with a first antibody, or a fragment thereof, covalently attached to a first oligonucleotide; (ii) contacting the biological sample with a second antibody, or a fragment thereof, covalently attached to a second oligonucleotide; (iii) contacting the biological sample with a signal-generating complex comprising a nucleic acid component capable of hybridizing to the first and second oligonucleotides; and (iv) detecting a signal from the signal-generating complex.

In some embodiments, the first antibody and/or the second antibody, or fragment thereof, is selected from a monoclonal antibody, a multispecific antibody, a bispecific antibody, a trispecific antibody, a tetravalent antibody, a single-domain antibody, a chimeric antibody, and a polyclonal antibody mixture. In some embodiments, the first antibody and/or the second antibody, or fragment thereof, is selected from a Fab, a scFv, a Fv, a scFv-Fc, a Fab′, a Fab′-SH, a F(ab′)₂, a diabody, a minibody, and a tribody.

In some embodiments, the first and/or the second oligonucleotide has a length of about 5 to about 100 nucleotides.

In some embodiments, step (i) and step (ii) are performed simultaneously. In some embodiments, step (i) is performed before step (ii). In some embodiments, step (ii) is performed before step (i).

In some embodiments, step (i) and/or step (ii) comprises contacting the biological sample with the first and/or second antibody for about 10 minutes to about 48 hours. In some embodiments, step (i) and/or step (ii) comprises contacting the biological sample with the first and/or second antibody at about 4° C. to about 75° C. In some embodiments, step (i) and/or step (ii) comprises contacting the biological sample with the first and/or second antibody at room temperature.

In some embodiments, the methods further comprise contacting the biological sample with a blocking agent before step (i) and/or step (ii). In some embodiments, the blocking agent comprises a protein, polypeptide, or nucleic acid.

In some embodiments, the methods further comprise contacting the biological sample with a crosslinking agent after steps (i) and (ii) but before step (iii). In some embodiments, the crosslinking agent is a fixative. In some embodiments, the methods comprise contacting the biological sample with the crosslinking agent for about 5 minutes to about 24 hours, at a temperature of about 4° C. to about 60° C.

In some embodiments, the methods further comprise contacting the biological sample with a protease after the crosslinking agent and before step (iii).

In some embodiments, the methods further comprise contacting the biological sample with a target probe set comprising a first target probe capable of hybridizing to the first oligonucleotide and to a section of the nucleic acid component of the signal-generating complex, and a second target probe capable of hybridizing to the second oligonucleotide and to a section of the nucleic acid component of the signal-generating complex.

In some embodiments, the first target probe comprises a target (T) section and a label (L) section, wherein the T section comprises a nucleic acid sequence complementary to a section of the first oligonucleotide and the L section comprises a nucleic acid sequence complementary to a section of the nucleic acid component of the second signal-generating complex. In some embodiments, the second target probe comprises a target (T) section and a label (L) section, wherein the T section comprises a nucleic acid sequence complementary to a section of the second oligonucleotide and the L section comprises a nucleic acid sequence complementary to a section of the nucleic acid component of the second signal-generating complex.

In some embodiments, the L sections are complementary to non-overlapping sections of the nucleic acid component of the second signal-generating complex. In some embodiments, the T sections are 3′ of the L sections. In some embodiments, the T sections are 5′ of the L sections. In some embodiments, the T sections are at least 5 nucleotides in length, and wherein the L sections are at least 5 nucleotides in length.

In some embodiments, the signal-generating complex comprises a pre-pre-amplifier, a pre-amplifier, and/or an amplifier; and one or more label probes, wherein each label probe comprises a detectable label. In some embodiments, the signal-generating complex comprises a pre-amplifier and an amplifier; and one or more label probes, wherein each label probe comprises a detectable label. In some embodiments, the detectable label comprises a fluorescent moiety or a chromogenic moiety. In some embodiments, the detectable label comprises a cleavable label.

In some embodiments, the methods further comprise (iv) contacting the biological sample with one or more third antibody, or a fragment thereof, and one or more fourth antibody, or a fragment thereof, wherein each of the third and fourth antibodies are covalently attached to an oligonucleotide; and (v) contacting the biological sample with one or more additional signal-generating complex comprising a nucleic acid component capable of hybridizing to the oligonucleotides covalently attached to the third and fourth antibodies.

In some embodiments, step (iv) and step (v) are performed simultaneously. In some embodiments, step (iv) and/or step (v) is performed before step (ii); step (iv) and/or step (v) is performed after step (ii); or step (iv) and/or step (v) is performed after step (iii).

In some embodiments, the methods further comprise contacting the biological sample with one of more nucleic acid detection agents.

In some embodiments, the biological sample is a tissue specimen or is derived from a tissue specimen. In some embodiments, the biological sample is a blood sample or is derived from a blood sample. In some embodiments, the biological sample is a cytological sample or is derived from a cytological sample. In some embodiments, the biological sample comprises cultured cells.

In some embodiments, the first antibody binds directly to an epitope on a first target protein, and the second antibody binds directly to an epitope on a second target protein. In some embodiments, the first antibody binds indirectly to an epitope on a first target protein, and the second antibody binds indirectly to an epitope on a second target protein. In some embodiments, the first antibody binds to an epitope on a first primary antibody that directly binds to an epitope on a first target protein, and the second antibody binds to an epitope on a second primary antibody that directly binds to an epitope on a second target protein.

In some embodiments, the first target protein and the second target protein are expressed on the surface of the same cell, and the signal produced from the signal generating complex indicates that the first target protein and the second target protein are in close proximity.

In some embodiments, the first target protein and the second target protein are expressed on the surface of different cells, and the signal produced from the signal generating complex indicates that the first target protein and the second target protein are in close proximity.

In some embodiments, the first antibody binds directly to a first epitope on a target protein, and the second antibody binds directly to a second epitope on the same target protein. In some embodiments, the first antibody binds indirectly to a first epitope on a target protein, and the second antibody binds indirectly to a second epitope on the same target protein. In some embodiments, the first antibody binds to a first primary antibody that directly binds to a first epitope on a target protein, and the second antibody binds to a second primary antibody that directly binds to a second epitope on the same target protein. In some embodiments, the signal produced from the signal generating complex indicates that the first epitope of the target protein and the second epitope of target protein are in close proximity.

In another aspect, the methods comprise: (i) contacting a biological sample with a first antibody, or fragment thereof, covalently attached to a first oligonucleotide, wherein the first antibody binds a first target epitope; (ii) contacting the biological sample with a second antibody, or fragment thereof, covalently attached to a second oligonucleotide, wherein the second antibody binds a second target epitope; (iii) contacting the biological sample with a pre-amplifier capable of hybridizing to the first and second oligonucleotides simultaneously, wherein the pre-amplifier comprises binding sites for a plurality of amplifiers; (iv) contacting the biological sample with the plurality of amplifiers capable of hybridizing to the pre-amplifier, wherein the plurality of amplifiers comprises binding sites for a plurality of label probes; (v) contacting the biological sample with the plurality of label probes capable of hybridizing to the plurality of amplifiers, wherein each label probe comprises a detectable label; and (vi) detecting a signal generated from the plurality of label probes when the first target epitope and the second target epitope are in sufficiently close proximity to allow binding of the pre-amplifier to the first and second oligonucleotides simultaneously.

In some embodiments, the methods further comprise contacting the biological sample with a target probe set after steps (i) and (ii). In some embodiments, the target probe set comprises a first target probe capable of hybridizing to the first oligonucleotide and to a section of the pre-amplifier, and a second target probe capable of hybridizing to the second oligonucleotide and to a section of the pre-amplifier. In some embodiments, the pre-amplifier is capable of hybridizing to the first and second target probes simultaneously.

In some embodiments, the first target probe comprises a target (T) section and a label (L) section, wherein the T section comprises a nucleic acid sequence complementary to a section of the first oligonucleotide and the L section comprises a nucleic acid sequence complementary to a section of the pre-amplifier. In some embodiments, the second target probe comprises a target (T) section and a label (L) section, wherein the T section comprises a nucleic acid sequence complementary to a section of the second oligonucleotide and the L section comprises a nucleic acid sequence complementary to a section of the pre-amplifier. In some embodiments, the L sections are complementary to non-overlapping sections of the nucleic acid component of the pre-amplifier.

In some embodiments, the first antibody binds directly to the first epitope, and the second antibody binds directly to the second epitope. In some embodiments, the first antibody binds indirectly to the first epitope, and the second antibody binds indirectly to a second epitope. In some embodiments, the first antibody binds to an epitope on a first primary antibody that directly binds to the first epitope. In some embodiments, the second antibody binds to an epitope on a second primary antibody that directly binds to the second epitope.

In some embodiments, the first epitope and the second epitope are on the same target protein. In some embodiments, the first epitope is on a first target protein and the second epitope is on a second target protein.

In some embodiments, the first target protein and the second target protein are expressed on the surface of the same cell and the signal produced from the signal generating complex indicates that the first target protein and the second target protein are in close proximity. In some embodiments, the first target protein and the second target protein are expressed on the surface of different cells and the signal produced from the signal generating complex indicates that the first target protein and the second target protein are in close proximity.

In another aspect provided herein are kits for detecting protein interactions in a biological sample. In some embodiments, the kits comprise: (i) a first antibody, or a fragment thereof, covalently attached to a first oligonucleotide, and a second antibody, or a fragment thereof, covalently attached to a second oligonucleotide; and (ii) a signal-generating complex, wherein the signal-generating complex comprises a nucleic acid component capable of hybridizing to the first and/or second oligonucleotide.

In some embodiments, the antibody or fragment thereof is selected from a monoclonal antibody, a multispecific antibody, a bispecific antibody, a trispecific antibody, a tetravalent antibody, a single-domain antibody, a chimeric antibody, and a polyclonal antibody mixture. In some embodiments, the antibody or fragment thereof is selected from a Fab, a scFv, a Fv, a scFv-Fc, a Fab′, a Fab′-SH, a F(ab′)₂, a diabody, a minibody, and a tribody.

In some embodiments, the oligonucleotide has a length of about 5 to about 100 nucleotides.

In some embodiments, the oligonucleotide is covalently attached to the antibody via a linker.

In some embodiments, the signal-generating complex comprises a pre-pre-amplifier, a pre-amplifier, and/or an amplifier; and one or more label probes, wherein each label probe comprises a detectable label. In some embodiments, the signal-generating complex comprises a pre-amplifier and an amplifier; and one or more label probes, wherein each label probe comprises a detectable label. In some embodiments, the detectable label comprises a fluorescent moiety or a chromogenic moiety.

In some embodiments, the kits further comprise a blocking agent, a crosslinking agent, a protease, or any combination thereof. In some embodiments, the kits further comprise instructions for carrying out a method of detecting the protein interactions in the biological sample.

In some embodiments, the kits further comprise a target probe set. In some embodiments, the target probe set comprises a first target probe capable of hybridizing to the first oligonucleotide and to a section of the nucleic acid component of the signal-generating complex. In some embodiments, the target probe set comprises second target probe capable of hybridizing to the second oligonucleotide and to a section of the nucleic acid component of the signal-generating complex.

In some embodiments, the first target probe comprises a target (T) section and a label (L) section, wherein the T section comprises a nucleic acid sequence complementary to a section of the first oligonucleotide and the L section comprises a nucleic acid sequence complementary to a section of the nucleic acid component of the signal-generating complex. In some embodiments, the second target probe comprises a target (T) section and a label (L) section, wherein the T section comprises a nucleic acid sequence complementary to a section of the second oligonucleotide and the L section comprises a nucleic acid sequence complementary to a section of the nucleic acid component of the signal-generating complex. In some embodiments, the L sections are complementary to non-overlapping sections of the nucleic acid component of the second signal-generating complex.

Other aspects and embodiments of the disclosure will be apparent in light of the following detailed description and accompanying figures.

### BRIEF DESCRIPTION OF THE DRAWINGS

FIG. 1A includes schematic illustrations of methods involving detecting one or more target proteins in a sample (right panel), as compared to traditional immunohistochemistry (left panel). FIG. 1B includes a schematic illustration of a representative workflow for the methods, which can include several optional steps (e.g., crosslink, protease treatment, and/or blocking steps).

FIG. 2A includes a schematic illustration of the various types of antibodies that can be used to detect a target antigen using the methods of the present disclosure, including but not limited to, monoclonal antibodies, multispecific antibodies, bispecific antibodies, trispecific antibodies, tetravalent antibodies, single-domain antibodies, chimeric antibodies, and a polyclonal antibody mixture. FIG. 2B includes a schematic illustration of an oligo-conjugated target antibody (e.g., monoclonal antibody, single-domain antibody) bound to a signal generating complex. FIG. 2C includes a schematic illustration of the use of multiple target antibodies (e.g., multiplexing).

FIGS. 3A and 3B show the use of an exemplary method as disclosed herein for detection of a PD-1/PD-L1 interaction. FIG. 3A is a schematic of an exemplary design comprising a first antibody to PD-1 and a second antibody to PD-L1, each comprising an oligonucleotide which associate to a signal generating complex. FIG. 3B shows images for detecting PD-1 and PD-L1 individually by IHC (top panels), and their proximity to each other using the methods described herein is shown in FIG. 3B, lower left. Negative controls using PD1 and PD-L1 antibodies conjugated to oligos show no detection when used individually, indicating the specificity of the detection method.

FIGS. 4A and 4B are images of the detection of CD3 subunits, CD3δ and CD3ε individually or using the methods disclosed herein. FIG. 4A, left column shows CD3δ detected using the signal generating complex associated with T1 channel (green), or T2 channel (red) detected individually, showing the individual marker positive populations in FFPE tonsil tissue. Right two columns indicate two combinations wherein CD3δ and CD3ε are detected only when they are in spatial proximity for the 5′ and 3′ oligonucleotide sequences to simultaneously build the signal generating complex. FIG. 4B shows positive control on left, with simultaneous detection of CD3δ-5′-T1 and CD3ε-3′-T1 positive cells, and corresponding negative controls using individual oligo-conjugated antibodies.

FIG. 5 is images of multiplexed detection of PD1-5′-T2/PD-L1-3′-T2 pair along with mRNA detection of Hs-IFNγ on lung cancer tissue sample. Shown in normal font are protein markers and in italics are mRNA markers assessed on the same slide.

FIG. 6A is a schematic illustration of 3-plex assay detecting individual proteins of PD-1 and PD-L1, and their interaction. FIGS. 6B and 6C are images of the 3-plex detection of PD-1/PD-L1/Interaction on Hodgkin's Lymphoma. Signal for interaction between PD-1 and PD-L1 (red) is detected only when PD-L1-positive Reed-Stenberg cells (magenta) and PD-1-positive signal from lymphocytes (green) are neighboring.

FIG. 7A is a low magnification image of multiplexed detection of PD1-PDL1 interaction, individual proteins (PanCK, CD107a, CD8a, CD4, CD3e) and mRNA (Hs-IFNG, Hs-GZMK, Hs-GZMB) on human bladder cancer tissue sample. The sample had few tumor-infiltrating CD3e-positive T lymphocytes. Lymphoid aggregates were observed between PanCK-positive tumor region. PD1-PDL1 interaction was observed within lymphoid aggregates but not within tumor on this sample. FIG. 7B is a higher magnification image of CD8a-positive (lime) lymphocyte-rich lymphoid aggregates on the same sample as FIG. 7A. The region shows mRNA ISH signals for Hs-IFNG (white), Hs-GZMK (green), Hs-GZMB (magenta). FIG. 7C is a higher magnification image of CD4-positive (blue) lymphocyte-rich lymphoid aggregates on the same sample as FIG. 7A. The region shows PD1-PDL1 interaction signals (red) within the aggregate.

FIG. 8A is a schematic illustration of integrin α_{V}β₁ heterodimer detection using a mix of primary and secondary antibodies. Integrin α_{V} (ITGAV) subunit is detected with a combination of rabbit anti-ITGAV primary antibody and anti-rabbit secondary antibody conjugated to 5′-T1 oligonucleotide. Integrin β₁ subunit is detected with anti-ITGB1 primary antibody conjugated to 3′-T1 oligonucleotide. Signal for heterodimer integrin α_{V}β₁ is observed when 5′-T1 and 3′-T1 oligonucleotides are in close proximity to build hybridize pre-amplifier. FIGS. 8B and 8C are images for chromogenic and fluorescent detection of integrin α_{V}β₁ heterodimer on mouse lung tissue. Left panels show heterodimer detected using signal generating complex (brown in chromogenic, red in fluorescent). Middle and right panels are negative controls that show no detection. Middle panel used rabbit anti-ITGAV primary antibody and anti-rabbit secondary antibody conjugated to 5′-T1 oligonucleotide. Right panel used anti-rabbit secondary antibody conjugated to 5′-T1 oligonucleotide and anti-ITGB1 primary antibody conjugated to 3′-T1 oligonucleotide.

### DETAILED DESCRIPTION

The present disclosure is directed to methods of detecting protein interactions in a biological sample and kits for conducting such methods. Rather than using traditional IHC methods, the methods disclosed herein use an antibody or a fragment thereof that is conjugated to an oligonucleotide for detection of a target protein(s). A signal-generating complex includes a nucleic acid component that can hybridize to the oligonucleotide, to provide a detectable signal that is indicative of a protein interaction or protein epitopes in close proximity. In some embodiments, the signal-generating complexes used in the methods disclosed herein provide highly amplified signals that enhance detection.

Section headings as used in this section and the entire disclosure herein are merely for organizational purposes and are not intended to be limiting.

### a. Definitions

As used in the present disclosure and claims, the singular forms “a,” “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “one or more” refers to, for example, 1 or more, 2 or more, 3 or more, 4 or more, 5 or more, 6 or more, 7 or more, 8 or more, 9 or more, 10 or more, 20 or more, 30 or more, 40 or more, 50 or more, or a greater number, if desired for a particular use.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

“Antibody” and “antibodies” as used herein refers to monoclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgAQ1, and IgA2), or subclass. For simplicity's sake, an antibody against an analyte is frequently referred to herein as being either an “anti-analyte antibody” or merely an “analyte antibody.”

“Antibody fragment” as used herein refers to a portion of an intact antibody that retain the ability to specifically bind to an antigen (see, generally, Holliger et al., Nat. Biotech., 23(9): 1126-1129 (2005)) (e.g., comprises the antigen-binding site or variable region). Any antigen-binding fragment of the antibody described herein is within the scope of the present disclosure. The antibody may not include the constant heavy chain domains (e.g., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the p sheets that provide the structural framework of the variable region (see, e.g., C. A. Janeway et al. (eds.), Immunobiology, 5th Ed., Garland Publishing, New York, N.Y. (2001)).

As used herein, the term “primary antibody” refers to an antibody that binds directly to the antigen of interest. As used herein, the term “secondary antibody” refers to an antibody that is conjugated to a moiety that can be used for detection, such as a detectable label, or a moiety to which a detectable label can bind. In some embodiments, a secondary antibody is conjugated to an oligonucleotide that is capable of hybridizing to a nucleic acid component of a signal-generating complex. In some embodiments, the secondary antibody provided herein binds directly to the primary antibody. In other embodiments, the secondary antibody provided herein binds indirectly to the primary antibody, e.g., by binding to another antibody that recognizes the primary antibody.

The term “monospecific” antibody as used herein denotes an antibody that has one or more binding sites each of which bind to the same epitope of the same antigen. The term “bispecific” antibody as used herein denotes an antibody that has at least two binding sites each of which bind to different epitopes of the same antigen or a different antigen. The term “multispecific” antibody as used herein denotes an antibody that has binding specificities for at least two different sites (e.g., bispecific, trispecific, tetraspecific).

The term “valent” as used within the current application denotes the presence of a specified number of binding sites in an antibody molecule. As such, the terms “bivalent,” “tetravalent,” and “hexavalent” denote the presence of two binding sites, four binding sites, and six binding sites, respectively, in an antibody molecule. The bispecific antibodies according to the invention are at least “bivalent” and may be “trivalent” or “multivalent” (e.g., “tetravalent” or “hexavalent”). That is, the antibodies may be bispecific even in cases where there are more than two binding sites (e.g., that the antibody is trivalent or multivalent).

As used herein, the term “close proximity” refers to two targets (e.g., target X and target Y) that are in physical or spatial proximity, either due to direct binding between the two targets or indirectly due to interactions between other molecules, cells, or the like. With regard to the methods disclosed herein, close proximity allows single detection of both targets with a single signal generating complex. In some embodiments, target X and target Y are on the same molecule (e.g., a protein). For example, target X and target Y may be different epitopes on a single protein and the targets are in “close proximity” to each other upon adopting a certain conformation indicative of, for example, a state of folding (or unfolding), binding to a substrate or ligand, activation, or post-translational processing. In some embodiments, target X and target Y are on different molecules. For example, target X and target Y are different molecules and are in “close proximity” when they are present in the same complex, bound to the same binding partner (e.g., protein, nucleic acid, small molecule, drug), in a similar location (e.g., on a cell membrane or in the same organelle), or on two associated structures or cells.

As used herein, the term “crosslink” refers to a process of binding two or more molecules together. The “crosslinking agent” or equivalent refers to agents containing two or more chemically reactive ends that attach themselves to the functional groups found in proteins and other molecules. Specifically, if the crosslinking agent is formaldehyde or its equivalent, a nucleophilic group on an amino acid or nucleic acid base forms a covalent bond with formaldehyde, which is stabilized in a second step that involves another functional group, often on another molecule, leading to formation of a methylene bridge. If the crosslinking agent is an oxidizing agent, it can react with the side chains of proteins and other biomolecules, allowing the formation of crosslinks that stabilize tissue structure.

The terms “detecting” as used herein generally refer to any form of measurement, and include determining whether an element is present or not. This term includes quantitative and/or qualitative determinations.

As used herein, the term “fixation” or “fixing” when made in reference to fixing a biological sample in the ISH process refers to a procedure to preserve a biological sample from decay due to, e.g., autolysis or putrefaction. It terminates any ongoing biochemical reactions and may also increase the treated tissues' mechanical strength or stability.

As used herein, the term “immunohistochemistry” or “IHC” generally refers to a technique for detecting proteins of interest in source samples utilizing antibodies, with the preservation of morphology of the source samples (e.g., tissue samples). As used herein, the term “immunocytochemistry” or “ICC” generally refers to a technique for detecting proteins of interest in source samples utilizing antibodies, with the preservation of morphology of the source samples (e.g., isolated or cultured intact cells, including tissue culture cell lines, either adherent or in suspension). Immunofluorescence (IF) refers to fluorescent labeling, thus it is also encompassed in the terms IHC and ICC. ICC, IHC, and IF assays can be used in conjunction with the imaging processing methods of the present disclosure, as described further herein, including facilitating quantitative and/or qualitative assessments of a target-of-interest in a sample. ICC, IHC, and IF assays can also be performed in conjunction with an in situ hybridization as part of an integrated co-detection process to detect targets-of-interest, which can also include performing the imaging processing methods of the present disclosure.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “protein interaction” as used herein refers to interactions within a single protein, or interactions between different proteins. The interactions may be due to direct binding (covalent or non-covalent) interaction or due to biochemical associations and processes which result in two different proteins or two locations in a single protein to be in close proximity. For example, protein interactions include, but are not limited to, interactions between protein components of a single multi-protein complex, protein binding pairs, two proteins binding the same target, two proteins localizing to a single location within a cell, two proteins on two different cells being in close proximity due to cell-cell interactions, two parts, subunits, or domains of a protein being in close proximity or non-covalently interacting as a result of folding, unfolding, activation, post-translational processing, binding of a target ligand or substrate, and the like.

The term “probe” as used herein refers to a capture agent that is directed to a specific target mRNA sequence. Accordingly, each probe of a probe set has a respective target mRNA sequence. In some embodiments, a probe can be used individually. In other embodiments, a probe can be used among a probe set. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, cells, and exosomes isolated from a mammal. Exemplary biological samples include but are not limited to cell lysate, a cell, a cell culture, a cell line, a tissue, oral tissue, gastrointestinal tissue, an organ, an organoid, a biological fluid, a blood sample, a urine sample, a skin sample, and the like. Preferred biological samples include, but are not limited to, whole blood, partially purified blood, PBMC, tissue biopsies, and the like.

### b. Methods of Detecting Protein Interactions in a Sample

In one aspect, provided herein are methods for detecting protein interactions in a biological sample. In some embodiments, the methods comprise: (i) contacting a biological sample with a first antibody, or a fragment thereof, covalently attached to a first oligonucleotide; (ii) contacting the biological sample with a second antibody, or a fragment thereof, covalently attached to a second oligonucleotide; (iii) contacting the biological sample with a signal-generating complex comprising a nucleic acid component capable of hybridizing to the first and second oligonucleotides; and (iv) detecting a signal from the signal-generating complex.

Steps (i) and (ii) involve use of antibodies or a fragment thereof, which are covalently attached to an oligonucleotide. The antibodies or the fragment thereof bind to a target protein in the sample, and their respective oligonucleotides provide a binding site for a signal-generating complex, as will be further discussed below.

Any suitable antibody can be used. In some embodiments, the first antibody and/or the second antibody, or fragments thereof, are selected from a monoclonal antibody, a multispecific antibody, a bispecific antibody, a trispecific antibody, a tetravalent antibody, a single-domain antibody, a chimeric antibody, and a polyclonal antibody mixture. In some embodiments, the first antibody and/or the second antibody, or fragments thereof, are selected from a Fab, a scFv, a Fv, a scFv-Fc, a Fab′, a Fab′-SH, a F(ab′)₂, a diabody, a minibody, and a tribody. In some embodiments, the first antibody and/or the second antibody may comprise a composition of polyclonal antibodies, in which a plurality of antibodies in the composition are conjugated to the oligonucleotide.

The oligonucleotides (e.g., first and/or the second oligonucleotide) that are covalently attached to the first antibody, the second antibody, or fragments thereof, can have a length of about 5 to about 100 nucleotides. In some embodiments, the first and/or the second oligonucleotide has a length of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In some embodiments, the first and/or the second oligonucleotide has a length of about 5 to about 50 nucleotides. In some embodiments, the first and/or the second oligonucleotide has a length of about 12 to about 16 nucleotides (e.g., 14 nucleotides). In some embodiments, the first and/or the second oligonucleotide has a length of about 26 to about 30 oligonucleotides (e.g., 28 nucleotides). In some embodiments, the first and/or the second oligonucleotide has a length of about 40 to about 60 nucleotides (e.g., 50 nucleotides). In some embodiments, the first and the second oligonucleotides are different in length. In some embodiments, the first and the second oligonucleotides have the same length.

As will be further discussed below, the sequence of the first and the second oligonucleotide is selected such that a nucleic acid component of the signal-generating complex is capable of hybridizing to the first and the second oligonucleotide. In some embodiments, the first and the second oligonucleotide has a sequence that is complementary to a sequence of a nucleic acid component of the signal-generating complex. For example, in some embodiments, the first and the second oligonucleotide has a sequence that is complementary to a sequence of a nucleic acid component of the signal-generating complex over a sequence of about 5 to about 100 nucleotides, e.g., a sequence of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In some embodiments, the first and the second oligonucleotide has a sequence that is complementary to a sequence of a nucleic acid component of the signal-generating complex over a sequence of about 5 to about 50 nucleotides, about 12 to about 16 nucleotides (e.g., 14 nucleotides, about 26 to about 30 oligonucleotides (e.g., 28 nucleotides), or about 40 to about 60 nucleotides (e.g., 50 nucleotides). In some embodiments, the portion of the first and second oligonucleotides which hybridize to the signal-generating complex hybridize or are complementary to non-overlapping sections of the nucleic acid component of the signal-generating complex.

The first and the second oligonucleotide is covalently attached to the first and second antibody or the fragment thereof, respectively. In some embodiments, the covalent attachment is via a direct bond between the antibody or the fragment thereof and the oligonucleotide. In some embodiments, the first and the second oligonucleotide is covalently attached via a linker.

General methods of conjugating oligonucleotides to antibodies are known to those skilled in the art. For example, a typical conjugation method includes use of a linker compound that includes two distinct reactive moieties, which react with different types of functional groups (e.g., one group that reacts with an amine, such as an activated ester group, and one group that reacts with a thiol, such as a maleimide group). Such reactive moieties used in conjugation reactions are well-known to those skilled in the art, and include activated esters such as succinimidyl and sulfosuccinimidyl esters and pentafluorophenyl esters, maleimides, azides, alkynes, hydrazines, isocyanates, isothiocyanates, haloacetamides, and the like. Methods of installing such reactive groups are well-known to those skilled in the art. As one non-limiting example, an amino group can be installed at the 5′-end of an oligonucleotide via phosphoramidite chemistry.

In some embodiments, an antibody is first reacted with the linker compound to provide a functionalized antibody, which is subsequently reacted with an oligonucleotide to provide the oligonucleotide-labeled antibody. In other embodiments, an oligonucleotide is first reacted with the linker compound to provide a functionalized oligonucleotide, which is subsequently reacted with an antibody to provide the oligonucleotide-labeled antibody.

Some oligonucleotide-antibody conjugation reagents or linkers are commercially available, and some are sold as parts of commercial kits. Exemplary commercially available linker compounds include those shown in Scheme 1, such as sulfosuccinimidyl-4-(N-maleimidomethyl)cyclohexane-1-carboxylate (sulfo-SMCC), succinimidyl-4-(N-maleimidomethyl)cyclohexane-1-carboxylate (SMCC), PEGylated crosslinkers such as SM(PEG)ₙ compounds (succinimidyl-([N-maleimidopropionamido](CH₂CH₂O)ₙ) esters), and 6-hydrazinonicotinate (HyNic) containing linkers such as S-HyNic.

In some embodiments, the commercial linkers can be used directly to conjugate the antibody to the oligonucleotide. In other embodiments, the antibody and/or the oligonucleotide must be first derivatized with a specific functional group prior to reaction with the linker compound. For example, the antibody can be reacted with a 2-iminothiolane to install a thiol group for reaction with a maleimide group. As another example, the oligonucleotide or antibody can be reacted with succinimidyl-4-formylbenzamide to install an aldehyde group for reaction with a HyNic-containing linker compound.

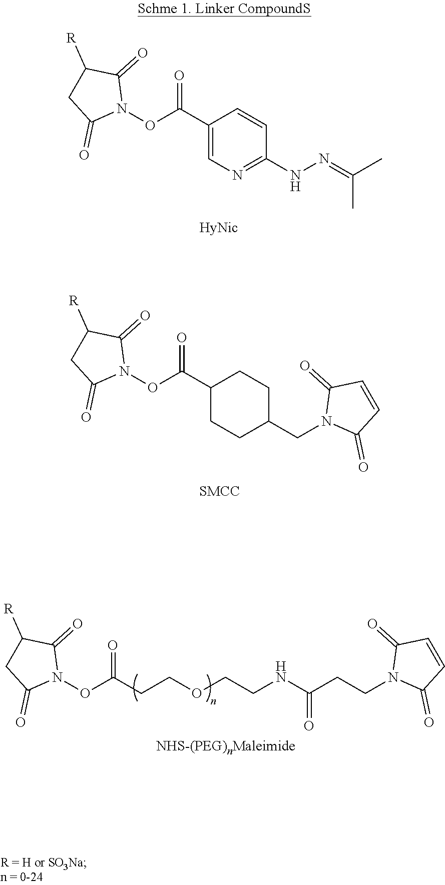

Other known linker chemistries involve separate functionalizations of the antibody and the oligonucleotide followed by a reaction to generate the linker moiety. Examples include installation of an azide-containing moiety on one compound and an alkyne-containing moiety on the other, for linkage via click chemistry (e.g., either copper-catalyzed or copper-free click chemistry).

Accordingly, in some embodiments, the linker comprises a moiety selected from:

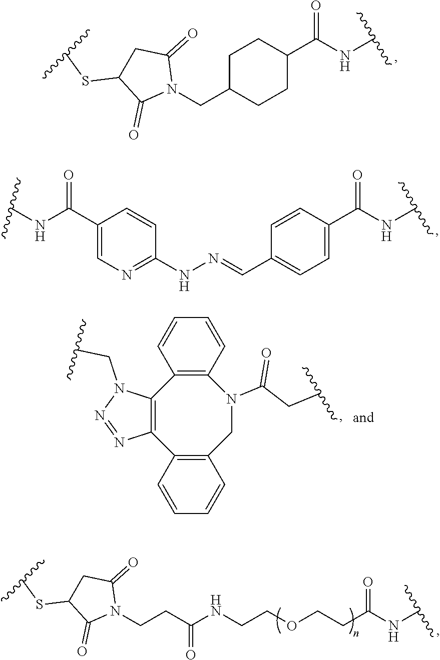

wherein n is 0, 1, 2, 3, 4, 5, 6, 7, 8, 9, 10, 11, 12, 13, 14, 15, 16, 17, 18, 19, 20, 21, 22, 23, or 24.

In some embodiments, the linker can include one or more nucleotides. For example, the linker can comprise an oligonucleotide sequence. Such a sequence may be considered separate from the oligonucleotide sequence to which the nucleic acid component of the signal-generating complex can hybridize. For example, in some embodiments, the linker can include one or more thymine groups. In some embodiments, the linker is a 5T linker.

The linker can include additional atoms or groups; for example, if the antibody is reacted with 2-iminothiolane, it is understood that the linker will further include atoms derived from such reaction. Accordingly, in some embodiments, the linker further comprises one or more additional groups selected from —CH₂—, —O—, —NH—, —S—, —C(═O)—, —C(═NH)—, and any combination thereof (e.g., combinations of such moieties could include ester groups (—C(═O)O—), amide groups (—C(═O)NH—), carbamate groups (—NHC(═O)O—), ethylene glycol groups (—CH₂CH₂O—), and the like.

In some embodiments, the linker comprises an antibody-binding domain. In some embodiments, an antibody binding domain (AbBD) comprises Protein A, Protein G, Protein L, CD4, or a fragment thereof. In some embodiments, the antibody-binding domain is an engineered antibody-binding domain, such as to include a non-natural amino acid, a photoreactive group, or a crosslinker. In some embodiments, the antibody binding domain is operably linked to a photoreactive amino acid group, for example, benzoylphenylalanine (BPA), resulting in a photoreactive antibody binding domain (pAbBD). In some embodiments, the antibody-binding domain (AbBD) is operably linked to a photoreactive amino acid which is operably linked to an antibody or a fragment thereof. See, for example, U.S. Pat. Nos. 11,156,608 and 11,123,440.

The method is not limited by the order in which steps (i) and (ii) are performed. In some embodiments, step (i) and step (ii) are performed simultaneously. In some embodiments, step (i) is performed before step (ii). In some embodiments, step (ii) is performed before step (i).

In some embodiments, step (i) and/or step (ii) comprises contacting the biological sample with the first and/or second antibody, or a fragment thereof, for about 10 minutes to about 48 hours, or about 15 minutes to about 120 minutes. For example, in some embodiments, step (i) and/or step (ii) comprises contacting the biological sample with the first and/or second antibody, or a fragment thereof, for about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, step (i) and/or step (ii) comprises contacting the biological sample with the first and/or second antibody or a fragment thereof at a temperature of about 4° C. to about 75° C., or about 4° C. to about 25° C. For example, in some embodiments, step (i) and/or step (ii) comprises contacting the biological sample with the first and/or second antibody or a fragment thereof at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, step (i) and/or step (ii) comprises contacting the biological sample with the first and/or second antibody or a fragment thereof at room temperature.

In some embodiments, the first and/or second antibody or a fragment thereof binds directly to the target in the biological sample. In such embodiments, the method does not require use of a primary antibody that binds directly to the protein. In other embodiments, the first and/or second antibody or a fragment thereof binds indirectly to the target protein in the biological sample. In such embodiments, the method can further comprise a step of contacting the sample with a first and/or second primary antibody prior to step (i) and/or step (ii), wherein the first and/or second primary antibody binds directly to a first and/or second target; and the first and/or second antibody or the fragment thereof that is covalently attached to an oligonucleotide then binds to the first and/or second primary antibody.

In some embodiments, the method further comprises contacting the sample with a blocking agent prior to step (i) and/or step (ii), to minimize non-specific binding that can result in unwanted background signals. Suitable blocking agents include those that comprise DNA, RNA, or protein. For example, in some embodiments, the blocking agent comprises DNA, such as salmon sperm DNA, herring sperm DNA, or calf thymus DNA. In some embodiments, the blocking agent comprises RNA, such as tRNA. In some embodiments, the blocking agent comprises a protein or polypeptide; for example, in some embodiments, the blocking agent comprises bovine serum albumin (BSA), casein, an animal serum such as normal goat serum, normal swine serum, normal chicken serum, or a fish serum such as steelhead salmon serum. In some embodiments, the blocking agent is a non-animal protein blocking agent, such as one comprising a plant protein. Non-animal protein blocking agents are commercially available, e.g., from G-Biosciences® (NAP-BLOCKER™) and Vector Laboratories (Animal-Free Blocker®).

In some embodiments, the method further comprises contacting the sample with a crosslinking agent after steps (i) and (ii) but before step (iii). Such a step has been found to preserve and even improve signals in IHC assays when conducted after incubation with a primary antibody and before incubation with a secondary antibody, such as when samples are exposed to protease treatment (see WO 2021/226311). In certain embodiments, the crosslinking agent is a fixative. In some embodiments, the crosslinking agent is selected from neutral-buffered formalin (NBF), formaldehyde, glutaraldehyde, acrolein, osmium tetroxide, a permanganate fixative (e.g., potassium permanganate), a dichromate fixative (e.g., potassium dichromate), chromic acid, and a mixture of any thereof. In particular embodiments, the crosslinking agent is NBF, such as about 1% to about 20% NBF (e.g., 10% NBF). In some embodiment, the crosslinking agent is a mixture of any of the above fixatives, with or without additional compounds. For example, in some embodiments, the crosslinking agent is selected from: Bouin's fixative (picric acid, formaldehyde, and acetic acid), a mixture of formaldehyde and glutaraldehyde; FAA (ethanol, acetic acid, and formaldehyde); periodate-lysine-paraformaldehyde (PLP) (paraformaldehyde, L-lysine, and INaO₄); phosphate buffered formalin (PBF); formal calcium (formaldehyde and calcium chloride); formal saline (formaldehyde and sodium chloride); zinc formalin (formaldehyde and zinc sulfate); Helly's fixative (formaldehyde, potassium dichromate, sodium sulphate, and mercuric chloride); Hollande's fixative (formaldehyde, copper acetate, picric acid, and acetic acid); Gendre's solution (formaldehyde, ethanol, picric acid, and glacial acetic acid); alcoholic formalin (formaldehyde, ethanol, and calcium acetate); and formol acetic alcohol (formaldehyde, glacial acetic acid, and ethanol). In some embodiments, the crosslinking agent comprises a polymer with at least two reactive functional groups, such as succinimidyl esters. In some embodiments, the crosslinking agent is a bis(succinimidyl) polyethylene glycol. In some embodiments, the crosslinking agent is provided as an aqueous solution at a pH of about 6 to about 9, e.g., about 6.0, about 6.5, about 7.0, about 7.5, about 8.0, about 8.5, or about 9.0. In embodiments in which two or more crosslinking agents are used, the sample may be contacted with the two or more crosslinking agents either simultaneously or consecutively.

In some embodiments, the step of contacting the sample with a crosslinking agent is conducted at a temperature of about 0° C. to about 100° C., about 1° C. to about 90° C., about 2° C. to about 80° C., about 3° C. to about 70° C., or about 4° C. to about 60° C., e.g., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 35° C., about 40° C., about 45° C., about 50° C., about 55° C., about 60° C., about 65° C., about 70° C., about 75° C., about 80° C., about 85° C., about 90° C., about 95° C., about or 100° C.

In some embodiments, the step of contacting the sample with a crosslinking agent is conducted for about 5 minutes to about 48 hours, about 5 minutes to about 24 hours, about 15 minutes to about 24 hours, or about 15 minutes to about 18 hours, e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, the method further comprises treating the biological sample with a protease after treating the biological sample with the crosslinking agent and before step (iii). This step can be used to digest certain proteins that surround the target. In some embodiments, the protease is selected from trypsin, proteinase K, pepsin, pronase, endoproteinase AspN, and endoproteinase GluC. In some embodiments, the method further comprises treating the biological sample with hydrogen peroxide after treating the biological sample with the crosslinking agent and before step (ii). This step is particularly useful when horseradish peroxidase (HRP) will be used as detection enzyme in the later steps, as the hydrogen peroxide inactivates endogenous HRP activity in the sample, thus reducing assay background.

Step (iii) of the method comprises contacting the sample with a signal-generating complex (SGC), wherein the signal-generating complex comprises a nucleic acid component capable of hybridizing to the first and second oligonucleotides. In some embodiments, the SGC is the same or similar SGC used in RNAscope™, which is described in more detail in, e.g., U.S. Pat. Nos. 7,709,198, 8,604,182, and 8,951,726. Specifically, RNAscope™ uses specially designed oligonucleotide probes in combination with a branched-DNA-like SGC to reliably detect RNA under standard bright-field microscopy (Anderson et al., J. Cell. Biochem. 117(10):2201-2208 (2016); Wang et al., J. Mol. Diagn. 14(1):22-29 (2012)). When used in methods described herein, rather than binding to one or more target probe(s) that bind to a target nucleic acid as in RNAscope™, the SGC instead binds to the oligonucleotides conjugated to the first and second antibodies or a fragment thereof.

In some embodiments, the SGC includes a pre-pre-amplifier, a pre-amplifier, and/or an amplifier, and one or more label probes, wherein each label probe comprises a detectable label. In some embodiments, the SGC comprises a pre-amplifier, an amplifier, and one or more label probes, wherein each label probe comprises a detectable label. Accordingly, the methods may comprise contacting the biological sample with a pre-pre-amplifier, a pre-amplifier, an amplifier, and/or one or more label probes simultaneously, sequentially in any order, or a combination thereof where some of the SGC components are provided simultaneously before or after another component or components.

Any nucleic acid portion of the SGC may hybridize to the to the first and second oligonucleotides, preferably simultaneously. In some embodiments, the pre-pre-amplifier hybridizes to the first and second oligonucleotides. In some embodiments, the pre-amplifier hybridizes to the first and second oligonucleotides. In some embodiments, the amplifier hybridizes to the first and second oligonucleotides.

The methods may further comprise: contacting the biological sample with a pre-amplifier capable of hybridizing to the first and second oligonucleotides simultaneously, wherein the pre-amplifier comprises binding sites for a plurality of amplifiers; contacting the biological sample with the plurality of amplifiers capable of hybridizing to the pre-amplifier, wherein the plurality of amplifiers comprises binding sites for a plurality of label probes; contacting the biological sample with the plurality of label probes capable of hybridizing to the plurality of amplifiers, wherein each label probe comprises a detectable label; and detecting a signal generated from the plurality of label probes when the first target epitope and the second target epitope are in sufficiently close proximity to allow binding of the pre-amplifier to the first and second oligonucleotides simultaneously.

Alternatively, the methods may further comprise: contacting the biological sample with a pre-pre-amplifier capable of hybridizing to the first and second oligonucleotides simultaneously, wherein the pre-pre-amplifier comprises binding sites for a plurality of pre-amplifiers; contacting the biological sample with a plurality of pre-amplifiers capable of hybridizing pre-amplifier simultaneously; contacting the biological sample with the plurality of amplifiers capable of hybridizing to the pre-amplifiers, wherein the plurality of amplifiers comprises binding sites for a plurality of label probes; contacting the biological sample with the plurality of label probes capable of hybridizing to the plurality of amplifiers, wherein each label probe comprises a detectable label; and detecting a signal generated from the plurality of label probes when the first target epitope and the second target epitope are in sufficiently close proximity to allow binding of the pre-amplifier to the first and second oligonucleotides simultaneously.

As used herein, an “amplifier” is a molecule, typically a polynucleotide, that is capable of hybridizing to multiple label probes. Typically, the amplifier hybridizes to multiple identical label probes. The amplifier can also hybridize directly to the target, or to another nucleic acid bound to the target such as a pre-amplifier. For example, the amplifier can hybridize to the target and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198, and U.S. Publication Nos. 2008/0038725 and 2009/0081688, each of which is incorporated herein by reference.

As used herein, a “pre-amplifier” is a molecule, typically a polynucleotide, that serves as an intermediate binding component between the target and one or more amplifiers. Typically, the pre-amplifier hybridizes simultaneously to the target and to a plurality of amplifiers. Exemplary pre-amplifiers are described, for example, in U.S. Pat. Nos. 5,635,352, 5,681,697, and 7,709,198, and U.S. Publication Nos. 2008/0038725, 2009/0081688 and 2017/0101672, each of which is incorporated by reference.

As used herein, a “pre-pre-amplifier” is a molecule, typically a polynucleotide, that serves as an intermediate binding component between the target and one or more pre-amplifiers. Typically, the pre-pre-amplifier hybridizes simultaneously to the target and to a plurality of pre-amplifiers. Exemplary pre-pre-amplifiers are described, for example, in U.S. Publication No. 2017/0101672, which is incorporated by reference.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. In general, the label probe can hybridize to a nucleic acid that is in turn hybridized to the target, or to one or more other nucleic acids that are hybridized to the target. Thus, the label probe can comprise a polynucleotide sequence that is complementary to a polynucleotide sequence, particularly a portion, of the target. Alternatively, the label probe can comprise at least one polynucleotide sequence that is complementary to a polynucleotide sequence in an amplifier, pre-amplifier, or pre-pre-amplifier in an SGC.

In some embodiments, the method further comprises contacting the biological sample with a target probe set comprising a first target probe capable of hybridizing to the first oligonucleotide and to a section of the nucleic acid component of the signal-generating complex, and a second target probe capable of hybridizing to the second oligonucleotide and to a section of the nucleic acid component of the signal-generating complex. As used herein, a “target probe” is a polynucleotide that is capable of hybridizing to the first or second oligonucleotide covalently attached to the first and second antibody and a component of the signal-generating complex (SGC). Accordingly, a “target probe set” comprises at least two polynucleotides: a first target probe capable of hybridizing to the first oligonucleotide covalently attached to the first antibody; and a second target probe capable of hybridizing to the second oligonucleotide covalently attached to the second antibody, each of the polynucleotides capable of hybridizing to the SGC.

A target probe (e.g., the first target probe or the second target probe) can hybridize directly to the label probe, or it can hybridize to one or more nucleic acids that in turn hybridize to the label probe; for example, a target probe can hybridize to an amplifier, a pre-amplifier, or a pre-pre-amplifier in an SGC. A target probe thus includes a first polynucleotide sequence that is complementary to a polynucleotide sequence of the first or second oligonucleotide and a second polynucleotide sequence that is complementary to a polynucleotide sequence of the label probe, amplifier, pre-amplifier, pre-pre-amplifier, or the like. A target probe is generally single stranded so that the complementary sequence is available to hybridize with a corresponding first or second oligonucleotide, label probe, amplifier, pre-amplifier, or pre-pre-amplifier.

In some embodiments, each target probe comprises a target (T) section and a label (L) section. Thus, the first target probe comprises a T section comprising a nucleic acid sequence complementary to a section of the first oligonucleotide and an L section comprising a nucleic acid sequence complementary to a section of the nucleic acid component of the second signal-generating complex; and the second target probe comprises a T section comprising a nucleic acid sequence complementary to a section of the second oligonucleotide and an L section comprising a nucleic acid sequence complementary to a section of the nucleic acid component of the second signal-generating complex.

The target probes are not limited by the orientation of the target (T) section with respect to the label (L) section. In some embodiments, the T sections are 3′ of the L sections. In some embodiments, the T sections are 5′ of the L sections. The T section and the L section may be consecutive or separated by any number of nucleotides to facilitate independent and accessible binding to the signal-generating complex and the oligonucleotides.

The target probes are also not limited by the length of the target (T) section with respect to the label (L) section. The length will be partially based upon the sequence and the number of nucleotides necessary for specific and/or selective binding to the signal-generating complex and the oligonucleotides. In some embodiments, the T sections are at least 5 nucleotides in length. For example, the T sections may be about 5, about 10, about 15, about 20, about 25, about 30, about 35, about 40, about 45, about 50, about 55, about 60, about 65, about 70, about 75, about 80, about 85, about 90, about 95, about 100, or more nucleotides in length. In some embodiments, the L sections are at least 5 nucleotides in length. For example, the L sections may be about 5, about 10, about 15, about 20, about 25, about 30, about 35, about 40, about 45, about 50, about 55, about 60, about 65, about 70, about 75, about 80, about 85, about 90, about 95, about 100, or more nucleotides in length. The length of the T and L sections may be the same or different. In some embodiments, the length of one of the T or L section may be longer, whereas the length of the other section may be shorter.

Both the first target probe and the second target probe bind to a nucleic acid component of the second signal-generating complex. The first target probe and the second target probe may bind the same nucleic acid component of the signal-generating complex. For example, the first target probe and the second target probe may both bind the label probe, amplifier, pre-amplifier, or pre-pre-amplifier. Alternatively, the first target probe and the second target probe may bind different nucleic acid components of the signal-generating complex.

In instances where the first and second target probes bind a single nucleic acid component of the signal-generating complex, the first and second target probes bind different locations within the nucleic acid component of the signal-generating complex. Thus, in some embodiments, the L sections of the first and second target probes are complementary to, and therefore hybridize to, non-overlapping sections of the nucleic acid component of the second signal-generating complex. In some embodiments, the nucleic acid component of the signal-generating complex is pre-amplifier. In some embodiments, the nucleic acid component of the signal-generating complex is pre-pre-amplifier.

In some embodiments, the target probe set comprises a first target probe capable of hybridizing to the first oligonucleotide and to a section of the pre-amplifier, and a second target probe capable of hybridizing to the second oligonucleotide and to a section of the pre-amplifier. In some embodiments, the pre-amplifier is capable of hybridizing to the first and second target probes simultaneously.

In some embodiments, the T section of the first target probe comprises a nucleic acid sequence complementary to a section of the first oligonucleotide and the L section of the first target probe comprises a nucleic acid sequence complementary to a section of the pre-amplifier. In some embodiments, the T section of the second target probe comprises a nucleic acid sequence complementary to a section of the second oligonucleotide and the L section of the second target probe comprises a nucleic acid sequence complementary to a section of the pre-amplifier. In some embodiments, the L section of the first target probe and the second target protein bind to non-overlapping sections of the pre-amplifier.

In some embodiments, the target probe set comprises a first target probe capable of hybridizing to the first oligonucleotide and to a section of the pre-pre-amplifier, and a second target probe capable of hybridizing to the second oligonucleotide and to a section of the pre-pre-amplifier. In some embodiments, the pre-pre-amplifier is capable of hybridizing to the first and second target probes simultaneously.

In some embodiments, the T section of the first target probe comprises a nucleic acid sequence complementary to a section of the first oligonucleotide and the L section of the first target probe comprises a nucleic acid sequence complementary to a section of the pre-pre-amplifier. In some embodiments, the T section of the second target probe comprises a nucleic acid sequence complementary to a section of the second oligonucleotide and the L section of the second target probe comprises a nucleic acid sequence complementary to a section of the pre-pre-amplifier. In some embodiments, the L section of the first target probe and the second target protein bind to non-overlapping sections of the pre-pre-amplifier.

As used herein, a “detectable label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In particular embodiments, the label comprises a fluorescent moiety or a chromogenic moiety. In a particular embodiment, the label is an enzyme. Exemplary enzyme labels include, but are not limited to horseradish peroxidase (HRP), alkaline phosphatase (AP), β-galactosidase, glucose oxidase, and the like, as well as various proteases. Other labels include, but are not limited to, fluorophores, dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837, 3,850,752, 3,939,350, 3,996,345, 4,277,437, 4,275,149, and 4,366,241. Many labels are commercially available and can be used in methods and assays of the disclosure, including detectable enzyme/substrate combinations (Pierce, Rockford IL; Santa Cruz Biotechnology, Dallas TX; Life Technologies, Carlsbad CA). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signal are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-nitrophenyl phosphate (PNPP) for alkaline phosphatase; 1-methyl-3-indolyl-β-D-galactopyranoside and 2-methoxy-4-(2-nitrovinyl)phenyl β-D-galactopyranoside for β-galactosidase; 2-methoxy-4-(2-nitrovinyl)phenyl β-D-glucopyranoside for β-glucosidase; and the like. Exemplary fluorogenic substrates include, but are not limited to, 4-(trifluoromethyl)umbelliferyl phosphate for alkaline phosphatase; 4-methylumbelliferyl phosphate bis(2-amino-2-methyl-1,3-propanediol), 4-methylumbelliferyl phosphate bis (cyclohexylammonium) and 4-methylumbelliferyl phosphate for phosphatases; QuantaBlu™ and Quintolet for horseradish peroxidase; 4-methylumbelliferyl β-D-galactopyranoside, fluorescein di(β-D-galactopyranoside) and naphthofluorescein di-(β-D-galactopyranoside) for β-galactosidase; 3-acetylumbelliferyl β-D-glucopyranoside and 4-methylumbelliferyl-β-D-glucopyranoside for β-glucosidase; and 4-methylumbelliferyl-α-D-galactopyranoside for α-galactosidase. Exemplary enzymes and substrates for producing a detectable signal are also described, for example, in U.S. Publication No. 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ⁵⁰Nd, ⁵¹Eu, ⁵²Sm, ⁵³Eu, ¹⁵⁴Sm, ⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb, ¹⁷²Yb, ¹⁷³Yb, ¹⁷⁴Yb, ¹⁷⁵Lu, and ¹⁷⁶Yb Metal isotopes can be detected, for example, using time-of-flight mass spectrometry (TOF-MS) (for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other, and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized horseradish peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

The methods of detecting protein interactions disclosed herein can be used for concurrent or sequential detection of multiple protein interactions in the same sample. For example, in some embodiments, the method comprises detecting two or more protein interactions in the same sample. In some embodiments, the method comprises detecting 2, 3, 4, 5, 6, 7, 8, 9, 10, 11, 12, 13, 14, 15, 16, 17, 18, 19, 20, or more different protein interactions in the same sample. For example, in some embodiments, the method comprises detecting from 1 to 100 different protein interactions in the sample. In some embodiments, the method comprises detecting from 1 to 50 different protein interactions in the sample.

For example, in some embodiments, the methods may comprise (iv) contacting the biological sample with one or more third antibody, or a fragment thereof, and one or more fourth antibody, or a fragment thereof, wherein each of the third and fourth antibodies are covalently attached to an oligonucleotide and (v) contacting the biological sample with one or more additional signal-generating complex comprising a nucleic acid component capable of hybridizing to the oligonucleotides covalently attached to the third and fourth antibodies. Accordingly, using one or more additional pairs of third and fourth antibodies allows detection of detecting two or more protein interactions in the same sample.

In some embodiments, step (iv) and step (v) are performed simultaneously. In some embodiments, step (iv) and/or step (v) is performed before step (ii); step (iv) and/or step (v) is performed after step (ii); or step (iv) and/or step (v) is performed after step (iii).

Embodiments in which higher numbers of protein interactions or other target molecules (e.g., nucleic acids) are detected in the same sample may involve the use of cleavable labels, which are further described below. In the case of using fluorophores as labels, the fluorophores to be used for detection of multiple protein interactions are selected so that each of the fluorophores are distinguishable and can be detected concurrently in a fluorescence microscope. Such fluorophores are selected to have spectral separation of the emissions so that distinct labeling of the target proteins can be detected concurrently. Methods of selecting suitable distinguishable fluorophores for use in methods of the disclosure are well known in the art (see, e.g., Johnson and Spence, “Molecular Probes Handbook, a Guide to Fluorescent Probes and Labeling Technologies, 11th ed., Life Technologies (2010)).

The label can be designed such that the labels are optionally cleavable. As used herein, a “cleavable label” refers to a label that is attached or conjugated to a label probe so that the label can be removed, for example, in order to use the same label in a subsequent round of labeling and detecting of targets. Methods for multiplex detection of nucleic acids of using cleavable labels have been described, e.g., in WO 2020/168162, which is incorporated herein by reference in its entirety, and are commercially available as RNAscope™ HiPlex assays (e.g., RNAscope™ HiPlex and RNAscope™ HiPlex v2).

Generally, the labels are conjugated to the label probe by a chemical linker that is cleavable. Methods of conjugating a label to a label probe so that the label is cleavable are well known to those skilled in the art (see, e.g., Hermanson, Bioconjugate Techniques, Academic Press, San Diego (1996); Daniel et al., BioTechniques 24(3):484-489 (1998)). One particular system of labeling oligonucleotides is the FastTag™ system (Daniel et al., supra, 1998; Vector Laboratories, Burlingame CA). Various cleavable moieties can be included in the linker so that the label can be cleaved from the label probe. Such cleavable moieties include groups that can be chemically, photochemically, or enzymatically cleaved. Cleavable chemical linkers can include a cleavable chemical moiety, such as disulfides, which can be cleaved by reduction, glycols, or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xa (cleavage site IEGR/), tobacco etch virus protease (cleavage site ENLYFQ/G), and thrombin (cleavage site LVPR/GS) (see, e.g., Oxford Genetics, Oxford, UK). Another cleavable moiety can be, for example, uracil-DNA (DNA containing uracil), which can be cleaved by uracil-DNA glycosylase (UNG) (see, e.g., Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

Step (iv) of the disclosed method comprises detecting a signal from the signal-generating complex. Well-known methods such as microscopy, cytometry (e.g., mass cytometry, cytometry by time of flight (CyTOF), flow cytometry), or spectroscopy can be utilized to detect chromogenic, fluorescent, or metal detectable signals associated with the respective targets. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of protein targets in the same sample.

The biological sample used in the disclosed methods can be derived from various sources. In one embodiment, the biological sample is a tissue specimen or is derived from a tissue specimen. In one embodiment, the biological sample is a blood sample or is derived from a blood sample. In one embodiment, the biological sample is a cytological sample or is derived from a cytological sample. In one embodiment, the biological sample is cultured cells. In another embodiment, the biological sample is a sample containing exosomes.

Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a target that is a biomarker indicative of a pathology.

The biological sample can be obtained from a subject, including a sample of biological tissue or fluid origin that is collected from an individual or some other source of biological material such as biopsy, autopsy, or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions, cells, and/or exosomes isolated from an organism such as a mammal. Exemplary biological samples include, but are not limited to, a cell culture, including a cell, a primary cell culture, a cell line, a tissue, an organ, an organoid, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, e.g., Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, e.g., Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In particular embodiments, the sample is a tissue specimen or is derived from a tissue specimen. In some embodiments, the tissue specimen is a formalin-fixed paraffin-embedded (FFPE) sample. In some embodiments, the tissue specimen is fresh frozen. In some embodiments, the tissue specimen is prepared with a fixative. In some embodiments, the tissue specimen is prepared with a crosslinking fixative. In other particular embodiments, the sample is a blood sample or is derived from a blood sample. In still other particular embodiments, the sample is a cytological sample or is derived from a cytological sample.

In some embodiments, the method further comprises steps to prepare a sample for detection of the target. For example, if the sample is a FFPE sample, a de-paraffinization step can be used to remove paraffin and rehydrate the sample. In some embodiments, the method further comprises dehydrating the biological sample. In certain embodiments, the dehydration is carried out with ethanol of increasing concentrations, such as in the order of 70%, 95%, and 100% ethanol.

In some embodiments, the method further comprises an epitope retrieval step, where certain epitope retrieval buffer(s) can be added to unmask the target. In some embodiments, the epitope retrieval step comprises heating the sample. In some embodiments, the epitope retrieval step comprises heating the sample to about 50° C. to about 100° C. In one embodiment, the epitope retrieval step comprises heating the sample to about 88° C. Detergents (e.g., Triton X-100 or SDS) and Proteinase K can be used to increase the permeability of the fixed cells. Detergent treatment, usually with Triton X-100 or SDS, is frequently used to permeate the membranes by extracting the lipids. Proteinase K is a nonspecific protease that is active over a wide pH range and is not easily inactivated. It is used to digest proteins that surround the targets. Optimal concentrations and durations of treatment can be experimentally determined as is well known in the art.

Any manner of protein interactions can be detected by the methods disclosed herein. The methods may detect interactions between two different epitopes of a single protein indicating they are in close proximity. The epitopes may be located on different subunits or domains of a single proteins, which change their proximity to each other during protein folding, protein processing, protein activation, or binding of a substrate or other ligand. Thus, the methods may be used to indirectly detect these protein biochemical events based on the proximity of the two epitopes. In some embodiments, the first antibody binds, directly or indirectly, to a first epitope on a target protein and the second antibody binds, directly or indirectly, to a second epitope on the same target protein. The first antibody and the second antibody do not need to bind to their respective epitopes by the same mechanism (e.g., directly or indirectly). For example, the first antibody may bind the first epitope directly, where the second antibody may bind the second epitope indirectly. Conversely, the first antibody may bind the first epitope indirectly, where the second antibody may bind the second epitope directly. In some embodiments, the first antibody binds to a first primary antibody that directly binds to a first epitope on a target protein. In some embodiments, the second antibody binds to a second primary antibody that directly binds to a second epitope on the same target protein.

The methods may detect interactions between two different proteins. For example, in some embodiments, the first antibody binds, directly or indirectly, to an epitope on a first target protein, and the second antibody binds, directly or indirectly, to an epitope on a second target protein. As described above in the context of a single protein, the first antibody and the second antibody do not need to bind to their respective protein targets by the same mechanism (e.g., directly or indirectly). In some embodiments, the first antibody binds to an epitope on a first primary antibody that directly binds to an epitope on a first target protein. In some embodiments, the second antibody binds to an epitope on a second primary antibody that directly binds to an epitope on a second target protein.

The two different proteins may be expressed by the same cell, thus detection of the protein interactions between the two protein indicates the two proteins are in close proximity. Close proximity may indicate, for example, that the two different proteins localized to similar structures in the same cell, within the same multi-protein complex, or associated with a common binding partner. In some embodiments, the first target protein and the second target protein are expressed on the surface of the same cell. The two different proteins may be expressed by different cells, thus detection of the protein interactions between the two protein indicates the two cells are in close proximity. In some embodiments, the first target protein and the second target protein are expressed on the surface of different cells.

In some embodiments, the methods comprise (i) contacting a biological sample with a first antibody, or fragment thereof, covalently attached to a first oligonucleotide, wherein the first antibody binds a first target epitope and (ii) contacting the biological sample with a second antibody, or fragment thereof, covalently attached to a second oligonucleotide, wherein the second antibody binds a second target epitope.

In some embodiments, the first epitope and the second epitope are on the same target protein. In some embodiments, the first epitope is on a first target protein and the second epitope is on a second target protein. In some embodiments, the first target protein and the second target protein are expressed on the surface of the same cell, and the signal produced from the signal generating complex indicates that the first target protein and the second target protein are in close proximity. In some embodiments, the first target protein and the second target protein are expressed on the surface of different cells, and the signal produced from the signal generating complex indicates that the first target protein and the second target protein, and thus the cells, are in close proximity.

In some embodiments, the methods further comprise (iii) contacting the biological sample with a pre-amplifier capable of hybridizing to the first and second oligonucleotides simultaneously, wherein the pre-amplifier comprises binding sites for a plurality of amplifiers; (iv) contacting the biological sample with the plurality of amplifiers capable of hybridizing to the pre-amplifier, wherein the plurality of amplifiers comprises binding sites for a plurality of label probes; (v) contacting the biological sample with the plurality of label probes capable of hybridizing to the plurality of amplifiers, wherein each label probe comprises a detectable label; and (vi) detecting a signal generated from the plurality of label probes when the first target epitope and the second target epitope are in sufficiently close proximity to allow binding of the pre-amplifier to the first and second oligonucleotides simultaneously.

The methods are not limited by the target proteins. Target proteins may include, but are not limited to: CD3d, CD3e, PD1, PD-L1, CTLA4, CD80/86, TIM3, Gal9/Ceacam1/HMGB1/PtdSer, TIGIT, CD112/CD155, PVRIG, PVRL2, LAG3, MHCII/Gal3/LSECtin/FGL1, CD27, CD70, CD40, CD40L, 4-IBB, 4-IBBL, OX40, OX40L, GITR, GITRL, ICOS, and ICOSL. In some embodiments, the methods disclosed herein can detect interaction between two target proteins, including, for example, CD3d and CD3e, PD1 and PD-L1, CTLA4 and CD80/86, TIM3 and Gal9/Ceacam1/HMGB1/PtdSer, TIGIT and CD112/CD155, PVRIG and PVRL2, LAG3 and MHCII/Gal3/LSECtin/FGL1, CD27 and CD70, CD40 and CD40L, 4-IBB and 4-IBBL, OX40 and OX40L, GITR and GITRL, and ICOS and ICOSL.

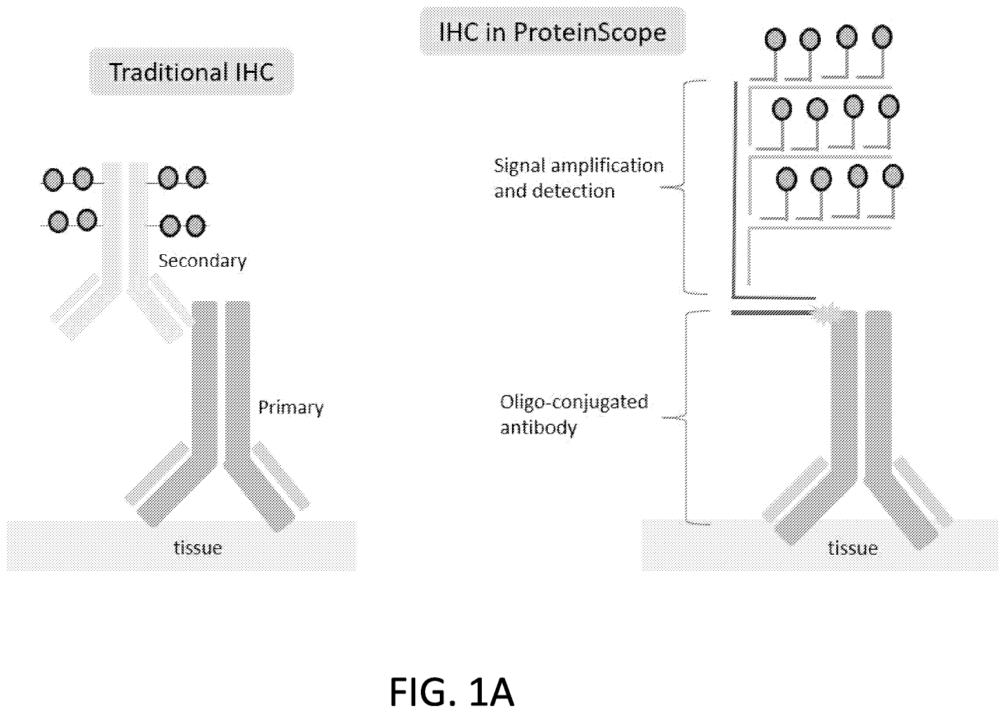

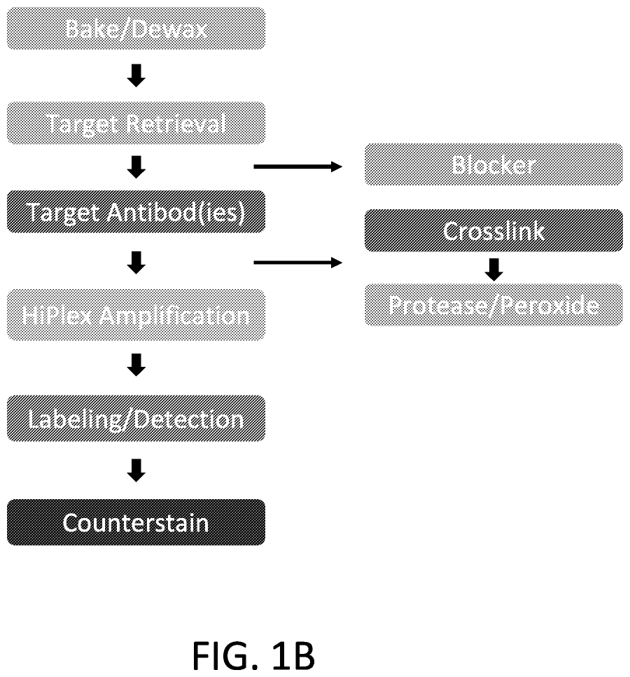

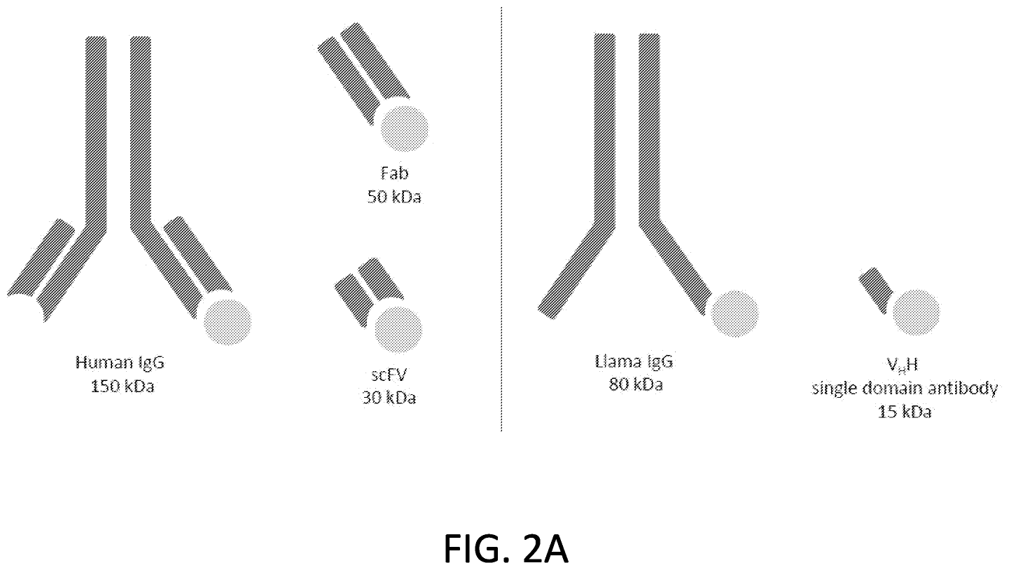

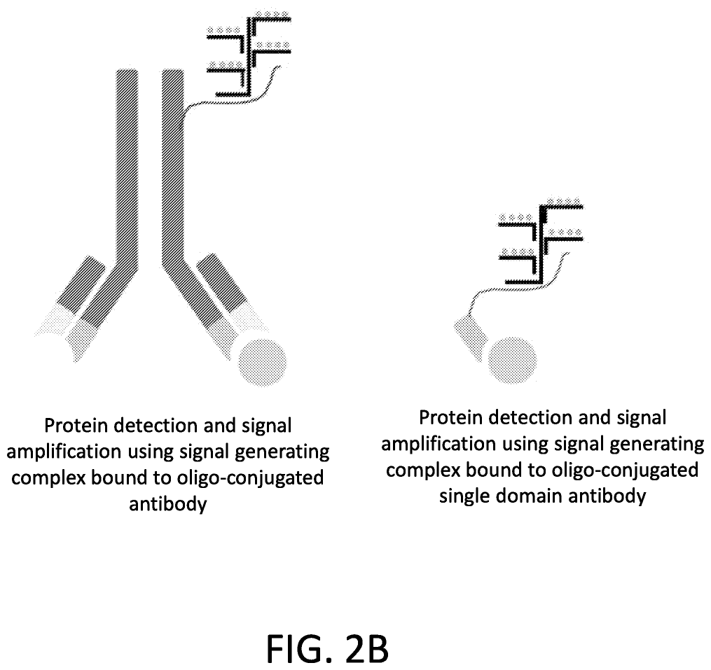

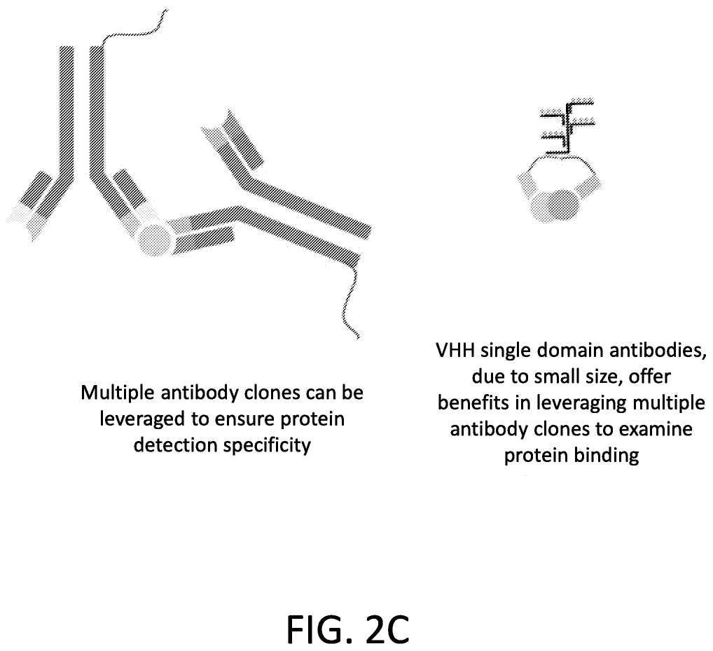

In some embodiments of the disclosed methods, an aptamer can be used in place of an antibody or fragment thereof. As used herein, the term “aptamer” refers to a single-stranded nucleic acid molecule (DNA or RNA) that can selectively bind to a specific target molecule, such as a target protein. In such embodiments, the oligo-conjugated antibody shown in the right panel of FIG. 1A would be replaced with an aptamer.

Accordingly, provided herein is a method for detecting protein interactions in a biological sample, comprising: (i) contacting a biological sample with a first aptamer covalently attached to a first oligonucleotide; (ii) contacting the biological sample with a second aptamer covalently attached to a second oligonucleotide; (iii) contacting the biological sample with a signal-generating complex comprising a nucleic acid component capable of hybridizing to the first and second oligonucleotides; and (iv) detecting a signal from the signal-generating complex.

In some embodiments, the aptamer has a length of about 10 nucleotides to about 100 nucleotides, or a length of about 20 nucleotides to about 60 nucleotides. In some embodiments, one of the more nucleotides in the aptamer is a modified nucleotide. As discussed above for the antibody-oligonucleotide conjugates, the aptamer can be directly attached to the oligonucleotide via a covalent bond. In such embodiments, the aptamer-oligonucleotide conjugate will comprise a single polynucleotide sequence, with one portion corresponding to the aptamer that binds to the target protein, and one portion corresponding to a sequence that can hybridize to a nucleic acid component of the signal-generating complex. In other embodiments, the aptamer sequence and the oligonucleotide sequence are separated by a linker, such as any linker disclosed herein.

The disclosed methods and components can also be used with methods and components for detection of other targets of interest in the sample. For example, the methods may further comprise detecting one or more nucleic acid targets. In some embodiments, the methods further comprise contacting the biological sample with one of more nucleic acid detection agents. The methods are not limited by the type of nucleic acid detection agents. For example, the nucleic acid detection agents may comprise in situ hybridization probes for specific sequences, or probes for detection of two or more nucleic acid targets (see, e.g., International Patent Publication WO2007001986).

### d. Kits

In yet another aspect, provided herein is a kit for performing the various methods described herein.

In one aspect, provided herein is a kit for detecting protein interactions in a biological sample, comprising: (i) a first antibody, or a fragment thereof, covalently attached to a first oligonucleotide, and a second antibody, or a fragment thereof, covalently attached to a second oligonucleotide; and (ii) a signal-generating complex, wherein the signal-generating complex comprises a nucleic acid component capable of hybridizing to the first and/or second oligonucleotide.

In another aspect, provided herein is a kit for detecting protein interactions in a biological sample, comprising: (i) a first and second oligonucleotide comprising a reactive moiety for conjugation to a first and second antibody; and (ii) a signal-generating complex, or nucleic acid components thereof (e.g., pre-pre-amplifier, a pre-amplifier, and/or an amplifier; and one or more label probes), wherein the signal-generating complex comprises a nucleic acid component capable of hybridizing to the first and/or second oligonucleotide. In some embodiments of such kits, the kit further comprises a conjugation reagent for conjugating the first and/or second oligonucleotide to an antibody (e.g., an antibody supplied separately from the kit).

In some embodiments, the kits further comprise a target probe set, wherein the target probe set comprises a first target probe capable of hybridizing to the first oligonucleotide and to a section of the nucleic acid component of the signal-generating complex, and a second target probe capable of hybridizing to the second oligonucleotide and to a section of the nucleic acid component of the signal-generating complex.

In another aspect, provided herein is a kit for detecting a protein interactions in a biological sample, comprising: (i) a first antibody, or a fragment thereof, covalently attached to a first oligonucleotide, and a second antibody, or a fragment thereof, covalently attached to a second oligonucleotide; (ii) one or more target probe(s) capable of hybridizing to the first and/or second oligonucleotide; and (ii) a signal-generating complex, or nucleic acid components thereof, capable of hybridizing to the one or more target probe(s), wherein the signal-generating complex comprises a nucleic acid component capable of hybridizing to the one or more target probe(s).

In another aspect, provided herein is a kit for detecting protein interactions in a biological sample, comprising: (i) a first antibody, or a fragment thereof, covalently attached to a first oligonucleotide, and a second antibody, or a fragment thereof, covalently attached to a second oligonucleotide; (ii) a first signal-generating complex, wherein the signal-generating complex comprises a nucleic acid component capable of hybridizing to the first and/or second oligonucleotide; (iii) one or more target probe(s) capable of hybridizing to the first and/or second oligonucleotide; and (iv) a second signal-generating complex capable of hybridizing to the one or more target probe(s), wherein the second signal-generating complex comprises a nucleic acid component capable of hybridizing to the one or more target probe(s).

The antibody or the fragment thereof, which is covalently attached to an oligonucleotide, is as described above. In the disclosed kit, the antibody, or the fragment thereof, the oligonucleotide, and the optional linker can be any of those described above. Similarly, the signal-generating complex(es), or nucleic acid components thereof, included in the kit can be any of those described above. In the kits that can include one or more target probe(s), the target probe(s) can be any of those described above.

In some embodiments, the kit further comprises a blocking agent, a crosslinking agent, a protease, or any combination thereof. The blocking agent, the crosslinking agent, and the protease can be selected from any of those described above.

The kit may further comprise packaging material, which refers to a physical structure housing the components of the kit. The packaging material can maintain the components in a sterile environment, and can be made of material commonly used for such purposes (e.g., paper, corrugated fiber, glass, plastic, foil, ampules, vials, tubes, etc.).

Kits provided herein can include labels or inserts, which can include information on a condition, disorder, disease, or symptom for which the kit component may be used for. Labels or inserts can include instructions for carrying out any of the methods disclosed herein. In some embodiments, labels or inserts can include instructions for a clinician or for a subject to use one or more of the kit components in a method, treatment protocol, or therapeutic regimen.

In some embodiments, the kit provided herein is used for mapping spatial organization in a complex tissue. In some embodiments, the kit provided herein is used for identifying cell types and new cell types. In some embodiments, the kit provided herein is used for identifying cellular states. In other embodiments, the kit provided herein is used for identifying cell types and new cell types in a tumor microenvironment. In some embodiments, the kit provided herein is used for identifying cellular states in a tumor microenvironment.

In some embodiments, the kit provided herein is used for identifying cell-cell interactions and new cell-cell interactions. In some embodiments, the kit provided herein is used for identifying cell-cell interactions and new cell-cell interactions in a tumor microenvironment. In some embodiments, the kit provided herein is used for studying tumor-immune cell interactions. In some embodiments, the kit provided herein is used for detecting biomarkers for cancer diagnosis and prognosis. In some embodiments, the kit provided herein is used for detecting therapeutic targets for cancer treatment. In some embodiments, the kit provided herein is used for facilitating the validation of novel antibodies.

### e. Image Processing

Embodiments of the present disclosure also include a method for enhancing detection of a protein interaction. In some embodiments, the method includes an image processing method. In some embodiments, the methods are implemented at least in part with a computer having corresponding instructions stored on a memory (e.g., a non-transitory computer readable medium). The final images, and in some embodiments the intermediate images, from the method are stored in a memory. In some embodiments, the memory is accessible by a network. In some embodiments, user input or instructions are receivable or accessible over the network.

The method includes imaging a sample with a target signal to create a probe image and imaging a sample with no target signal to create a background image (e.g., “blank image”). In some embodiments, the imaging utilizes a fluorescent microscope coupled to a computer via a network. In some embodiments, the target signal is obtained by subjecting the sample to a method disclosed herein. In some embodiments, the background image with no target signal is obtained by removing the signal from the sample (i.e., by a cleaving process). In other embodiments, the background image with no target signal is obtained before the assay is performed.

The method includes registering the assay image and the background image. Potential background fluorescence discrepancy between the assay image and the background image creates spatial pattern mismatches that occur due to whole sample movement between different rounds of image acquisition. To remove such discrepancies, image registration techniques (e.g., phase correlation) are utilized. Robust image registration utilized detection and matching of image features to compensate for any global sample movement (e.g., translation and rotation).

The method further includes modifying the background image to create an adjusted background image (e.g., transformed, intensity-adjusted blank image) based on at least one image metric. The at least one image metric may be a ratio factor (to account for intensity differences in background between the blank image and the probe image), a multiplication factor (to account for potential local intensity differences between the blank image and the probe image), a local maximum value transform (to account for local background pattern mismatches that are from, for example, image acquisition at different focal planes, or samples not firmly attached to the supporting material), a block-matching transform (to resolve the issue of local mismatches) and any other suitable metric. In some embodiments, the method includes a single image metric. In other embodiments, the method includes a combination of image metrics.

The method further includes subtracting the adjusted background image from the assay image to create a final image comprising an enhanced target signal. In other words, the modified (e.g., transformed, adjusted, scaled, etc.) blank image is used in the subtracting step instead of the original blank image. In some embodiments, the enhanced target signal includes enhanced contrast. In some embodiments, the method includes displaying the final on a display (e.g., a computer display). The final image may be saved to a memory and may be accessible by a user, for example, over a network. As such, the method provides improved signal detection in the presence of a background with tissue autofluorescence.

In some embodiments, the method for enhancing detection includes any combination of the steps described herein, in various orders. In some embodiments, steps may be omitted. Further, the order of the steps may be reversed, altered, or performed simultaneously.

In at least one embodiment, the electronic-based aspects of the method may be implemented in software (e.g., stored on non-transitory computer-readable medium) executable by a computer with one or more processing units, such as a microprocessor and/or application specific integrated circuits (“ASICs”). Some embodiments may include hardware, software, and electronic components or modules. As such, it should be noted that a plurality of hardware and software-based devices, as well as a plurality of different structural components, may be utilized to implement the embodiments.

### EXAMPLES

The following is a description of methods, materials, and results corresponding to the various embodiments of the present disclosure. These descriptions are provided as examples and are not intended to be limiting. Rather, these examples are intended to provide those of ordinary skill in the art with a description of how to make and use the various embodiments of the present disclosure. These examples are not intended to limit the scope of what the inventors regard as inventive subject matter, nor are they intended to represent all of the experiments that can be performed. It is to be understood that exemplary descriptions written in the present tense were not necessarily performed, but rather that the descriptions can be performed to generate the data associated with the teachings of the present disclosure. Efforts have been made to ensure accuracy with respect to numbers used (e.g., amounts, percentages, etc.), but some experimental error and deviation may be present.

### Materials and Methods

Design of oligonucleotide for conjugation to antibody. For protein-protein co-detection, a pair of single stranded DNA oligonucleotides, labeled 5′-Tx and 3′-Tx (where 1<=x<=12), are designed such that the two sequences when bound in proximity, together serve as the pre-amplifier binding site (RNAscope HiPlex v2 Amp 1, Advanced Cell Diagnostics) for subsequent amplification. The binding of pre-amplifier to the pair of sequences together prevents non-specific binding to any single oligonucleotides that may bind randomly, so that signals are generated only when the pair is in proximity with correct spatial arrangement. While any oligo-antibody conjugation method that retains antibody functionality can be used, described herein is one site-specific conjugation technique.

Primary antibody-oligonucleotide conjugation. Primary antibodies targeting the protein of interest and its potential protein pair, are each mixed with stock oYo-Link®-modified oligonucleotides (provided at 33 μM), at antibody:oligo molar ratio of 1:5 in separate transparent microcentrifuge tubes (one protein and one oligo per tube). The tube is then placed inside a photo-crosslinking device (LED-PX, AlphaThera) for 4 hours at 4° C. oYo-Link is activated with ultraviolet light at 365 nm wavelength for conjugation to the antibody (James Z. Hui, alphathera.com).

Purification of antibody-oligonucleotide conjugate. After 4 hours of conjugation, the solution contains antibody-oligonucleotide conjugates, unconjugated (free) antibody, and free oYo-Link-modified oligonucleotides. The mixture can be purified by using a molecular weight cutoff (MWCO) filtration system. In this method, Amicon Ultra Centrifugal Filter 0.5 ml Units of MWCO at 30 kDa or 100 kDa (Millipore-Sigma) is used where applicable. Manufacturer recommendations were modified for purification. Briefly, the conjugation solution is added to the filtration device placed in a microcentrifuge tube, and made up to 500 μL total volume with phosphate buffered saline (PBS) or tris buffered saline (TBS). The tube containing the filtration device is then centrifuged at 14000×g for 5 min (30 kDa) or 10 min (100 kDa). After discarding the filtrate in the bottom compartment, PBS or TBS is again added to 500 μL total volume in the filtration device, then centrifuged at the same speed/time. Following this, a reverse spin is performed at 1000×g for 2 min with the filtration device inserted upside down in a new microcentrifuge tube, for recovery of purified antibody-oligonucleotide conjugate.

Formalin-fixed paraffin-embedded (FFPE) tissue preparation (manual assay). The assay can be performed both as a manual assay or semi-automated on platforms such as the Leica BOND RX System. Described herein are steps used in the manual assay. To deparaffinize formalin-fixed paraffin-embedded (FFPE) tissue section, the slide with FFPE tissue section is baked at 60° C. for 30 min to 1 hour. Slides are then transferred to fresh Xylene for 5 min incubation, followed by another 5 min incubation in fresh Xylene. Next, slides are immersed in fresh 100% ethanol for 2 min and repeated with fresh 100% ethanol. Slides are then dried at 60° C. for 5 min or at room temperature for overnight. Endogenous peroxidases in tissue are quenched by applying 3% hydrogen peroxide for 10 min at room temperature. Next, target retrieval is performed by immersing the slides in Co-Detection Target Retrieval reagent (Advanced Cell Diagnostics, Newark, CA) for 15 min at 100° C., then rinsed in deionized water twice followed by rinsing with PBS.

Incubation of antibody-oligonucleotide conjugates to protein targets on tissue (manual assay). Target-retrieved slides are blocked with 500 μg/ml fish sperm DNA (Cat #AM9680, Invitrogen) diluted in Co-Detection Antibody Diluent (Cat #323160, Advanced Cell Diagnostics, Newark, CA) (or other antibody diluent optimized for detection) for 1 h at room temperature. Slides are then briefly washed with 0.5% Tween-20 in PBS (PBST). Oligonucleotide-conjugated antibodies against the desired targets are diluted in the appropriate antibody diluent to desired final concentrations. Antibody cocktail is then applied on the slides and incubated for 1 hour at room temperature. Slides are washed twice in FFPE wash buffer (Cat #210091, Advanced Cell Diagnostics, Newark, CA) for 2 min. Bound antibodies are then cross-linked by incubating the slides in neutral buffered formaldehyde (NBF). Slides are extensively washed 4 times in 1×FFPE wash buffer for 2 min, followed by application of Protease III (Cat #322327, Advanced Cell Diagnostics, Newark, CA) for 15 min at 40° C. using HybEZ™ II oven.

Signal amplification. For this step, RNAscope HiPlex12 Detection Kit v2 (Cat #324400 or 324410, Advanced Cell Diagnostics, Newark, CA) is used. Slides are incubated with RNAscope HiPlex Amp 1 for 30 min at 40° C. in HybEZ™ II oven, followed by two washes for 2 min with 1×FFPE wash buffer. Next, RNAscope HiPlex Amp 2 is applied to the slides and incubated for 30 min at 40° C. in HybEZ™ II oven, followed by two washes for 2 min with 1×FFPE wash buffer. Slides are then incubated with RNAscope HiPlex Amp 3 for 30 min at 40° C., and subsequently washed twice with 1×FFPE wash buffer. To quench tissue autofluorescence, FFPE reagent diluted in 4×SSC (1:20-1:40 dilution), is applied to the slides, and incubated at room temperature for 30 min. After washing with 1×FFPE wash buffer, RNAscope HiPlex Fluoro T1-T4 v2 is applied to the slides and incubated at 40° C. for 15 min. Following another wash with 1×FFPE wash buffer, slides are incubated with DAPI for 30 s at room temperature. After removing excess DAPI, slides are mounted using ProLong Gold Antifade Mountant (Invitrogen), coverslipped, and imaged under fluorescent microscope or scanner.

Following imaging of round 1 (T1-T4) markers, the assay is continued for detection of T5-T8 channels. To facilitate application of subsequent reagents, coverslips are removed from slides by soaking them in 4×SSC. After coverslips are removed, slides are briefly washed in fresh 4×SSC. T1-T4 fluorophores are cleaved by incubating the slides with 10% cleaving solution v2 (Advanced Cell Diagnostics) diluted in 4×SSC for 15 min at room temperature. Excess cleaving solution is removed and washed twice in PBST. Another round of fluorophore cleaving is repeated as described previously. Next, RNAscope HiPlex Fluoro T5-T8 v2 is applied and slides are incubated at 40° C. for 15 min. This is followed by two washes with 1×FFPE wash buffer for 2 min each. Slides are then mounted and coverslipped for imaging. To image targets in T9-T12 channels, the same process of cleaving and application of RNAscope HiPlex Fluoro T9-T12 v2 at 40° C. for 15 min is followed.

### Example 1

Immune checkpoint inhibitory receptors, such as cytotoxic T lymphocyte antigen 4 (CTLA4) and programmed cell death protein 1 (PD-1), expressed on immune cells trigger immunosuppressive signaling pathways. For example, PD-1 binds to PD-L1 or PD-L2 and resists positive signals through T-cell receptors (TCRs) and CD28. In general, expression of PD-L1 is observed on T, B, and antigen-presenting cells and in some non-lymphoid tissues. Ligand binding to PD-1 on the surface of T cells mediates immune inhibition. In addition, PD-L1 is detected in the cardiac endothelium, placenta, and pancreatic islets, which indicates a role of PD-L1 in immunological tolerance.

These immunosuppressive molecules function as brakes to regulate the adaptive immune response. Their use has also been translated to the clinic. Recent years have witnessed the wide application of anti-PD-1 and anti-PD-L1 antibodies in various types of cancer. The PD-1/PD-L1 interaction inhibits T lymphocyte proliferation, survival, and effector functions (cytotoxicity, cytokine release), induces apoptosis of tumor-specific T cells, promotes the differentiation of CD4⁺ T cells into Foxp3⁺ regulatory T cells, as well as the resistance of tumor cells to CTL attack.

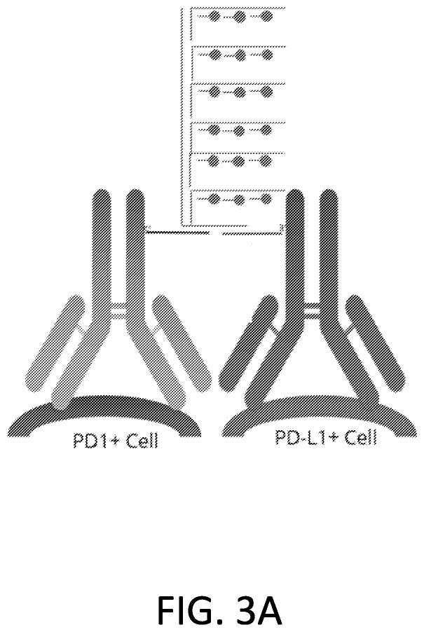

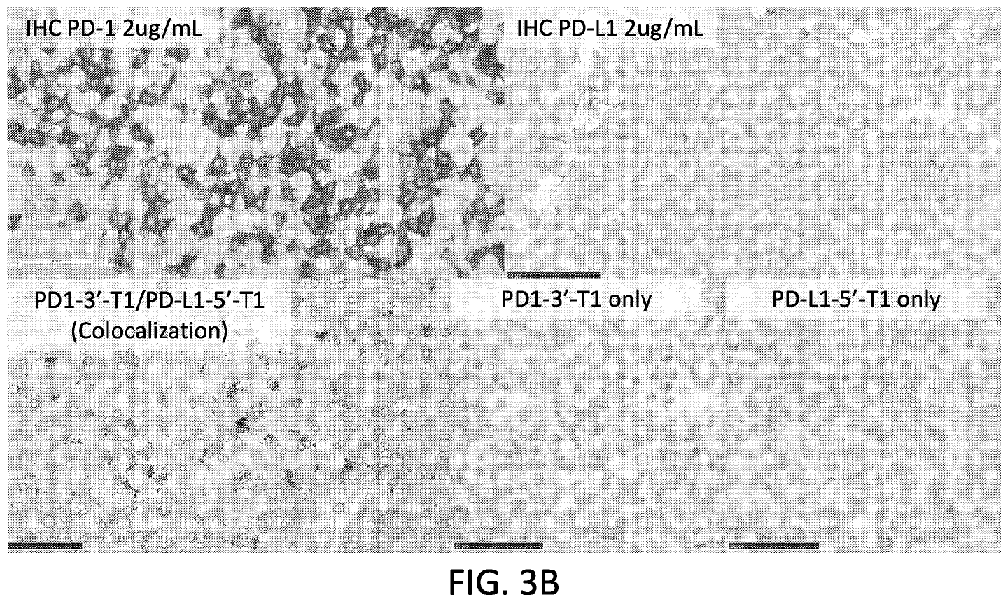

As shown in the lower left image of FIG. 3B, colocalization representing the proximity of PD1 to PD-L1 is able to be detected using the methods disclosed herein. Negative controls using PD1 and PD-L1 antibodies conjugated to oligos show no detection when used individually, indicating the specificity of the detection method.

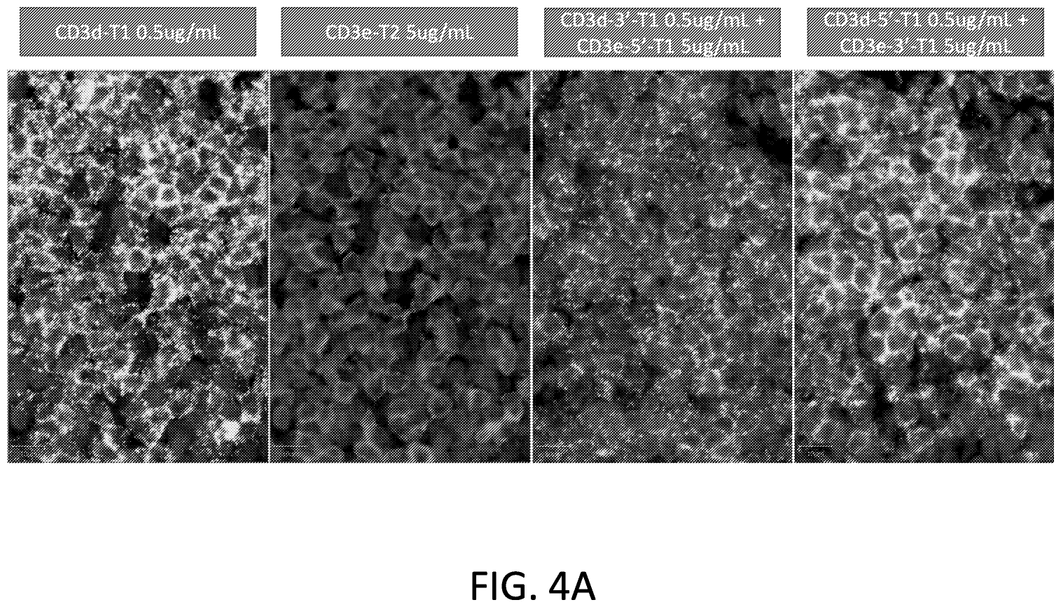

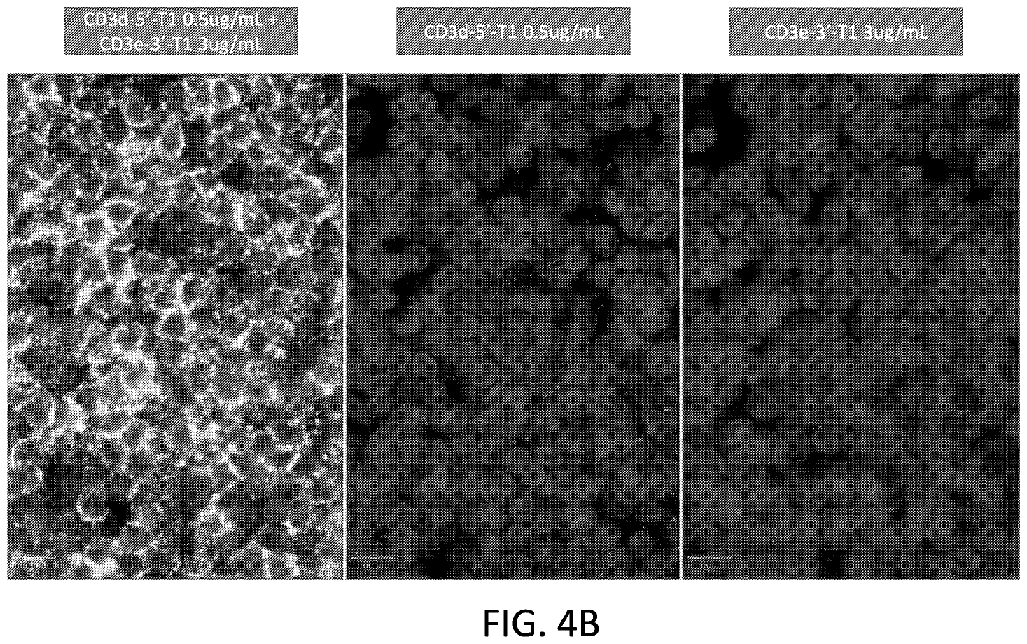

CD3 is a membrane marker for T-cells comprising multiple subunits. Specifically, CD3ε interacts with CD3γ as well as CD3δ. As shown in FIGS. 4A and 4B, detection of spatial proximity CD3 subunits, CD3δ and CD3ε, can be accomplished using the methods disclosed herein.

### Example 2

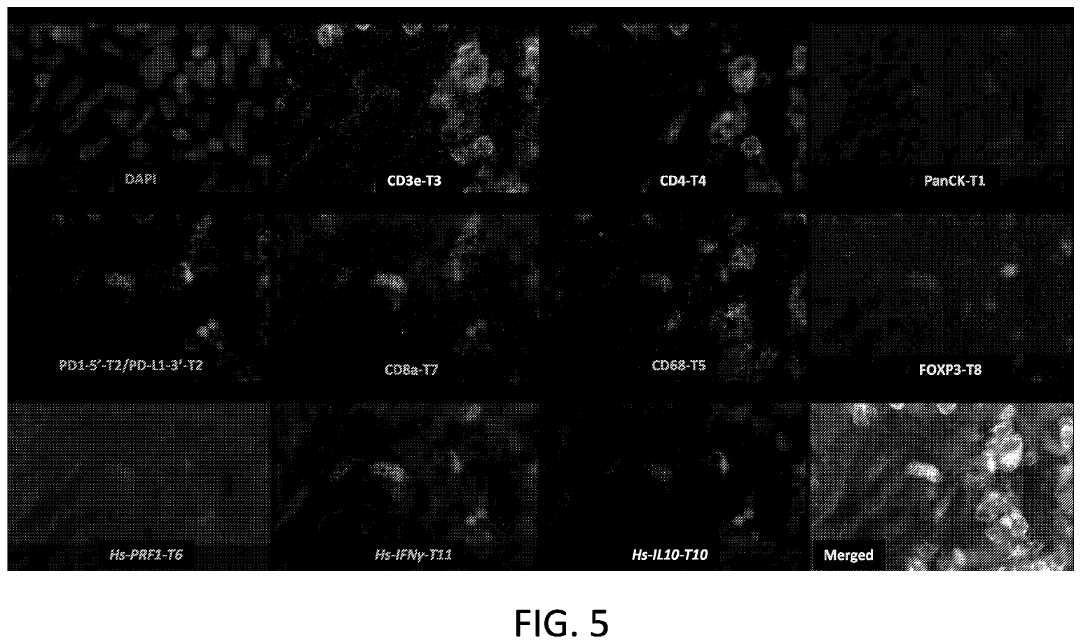

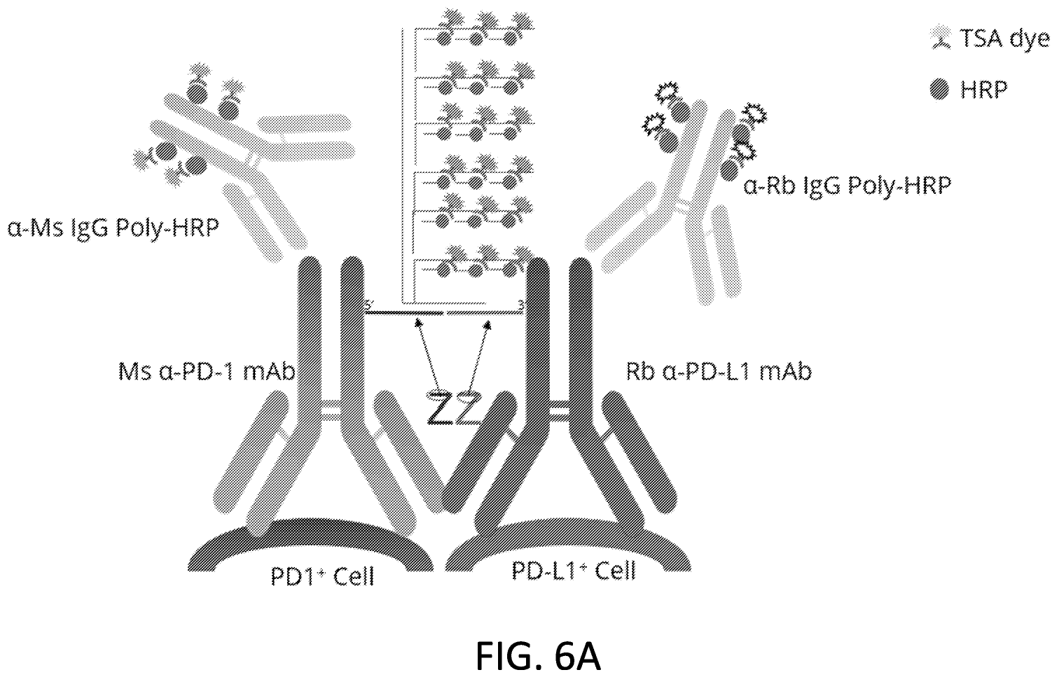

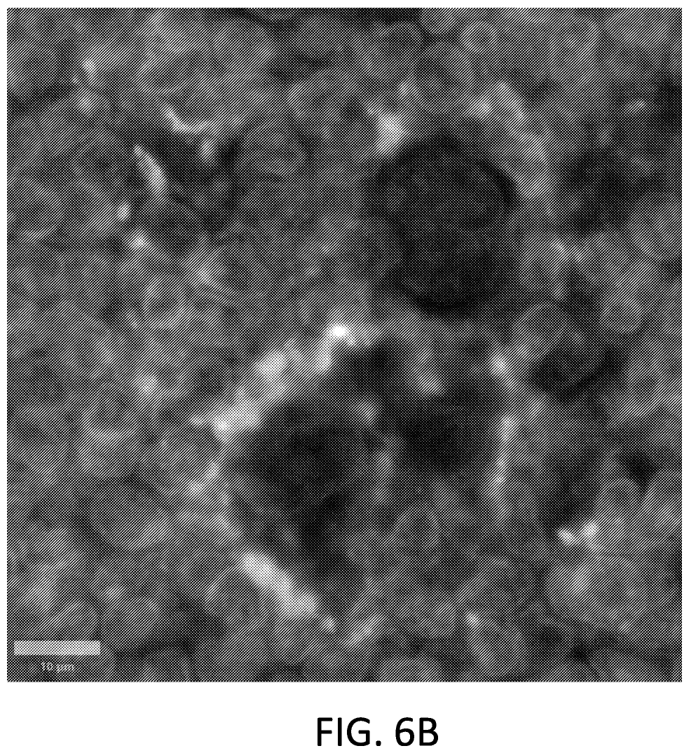

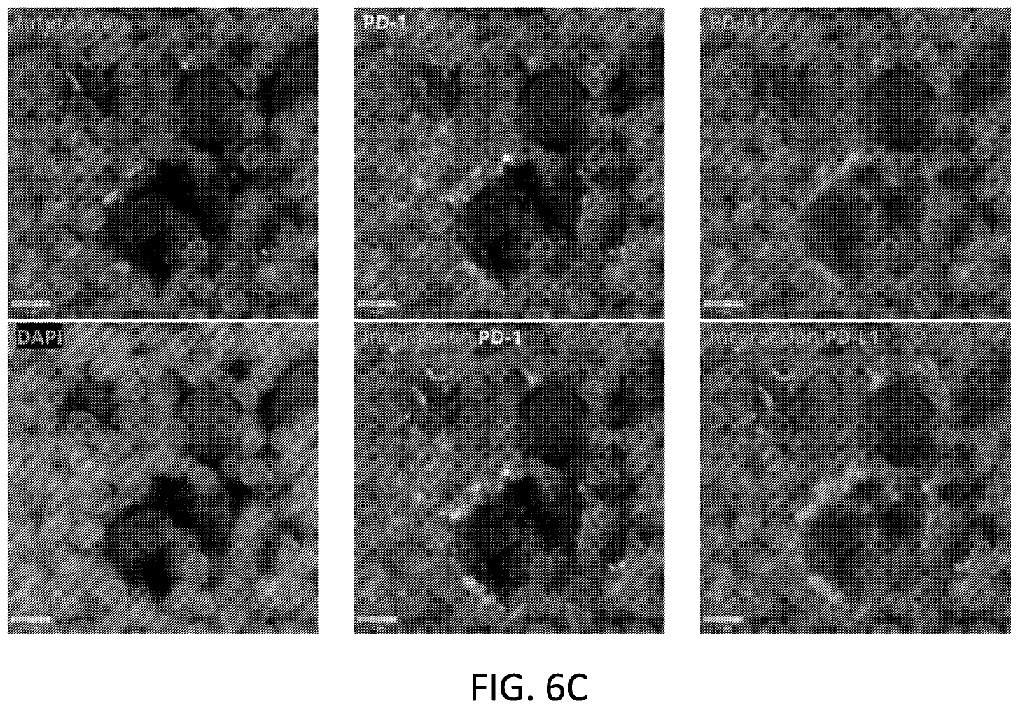

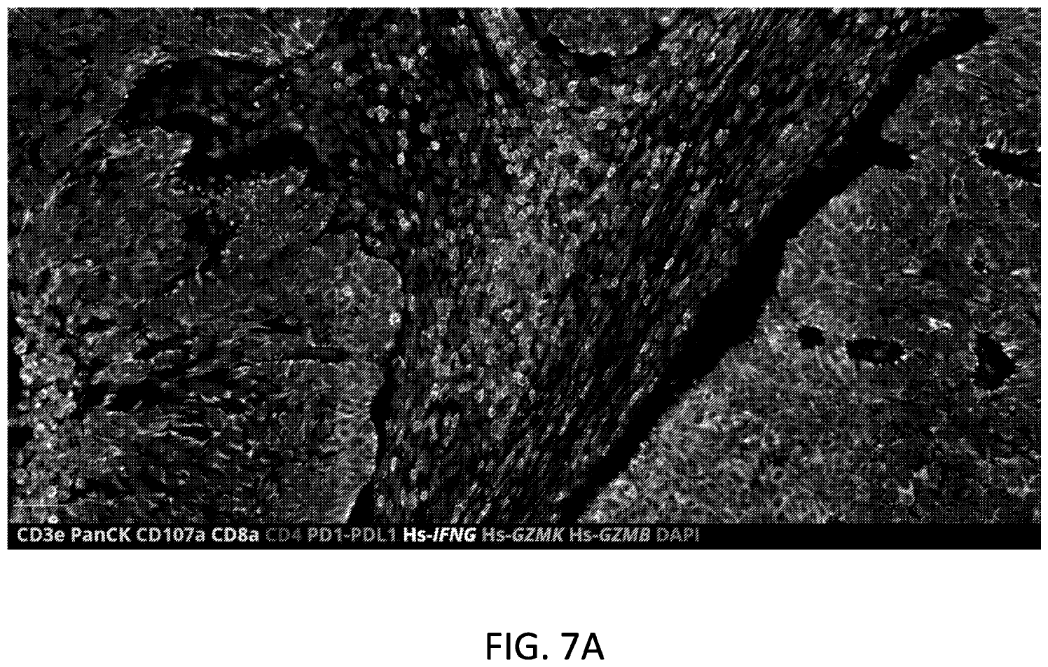

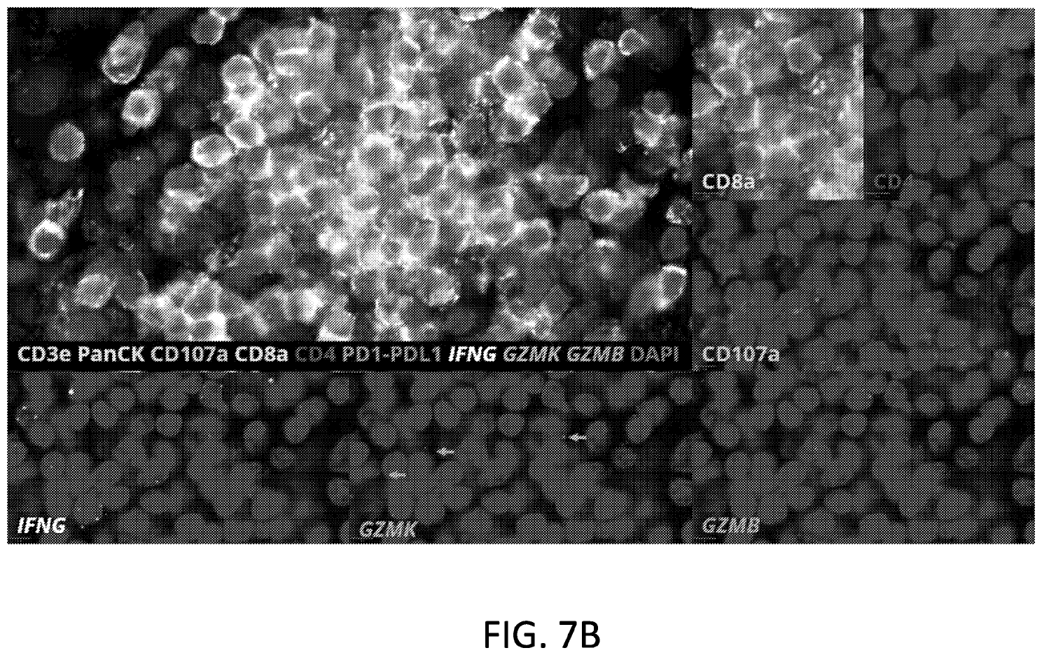

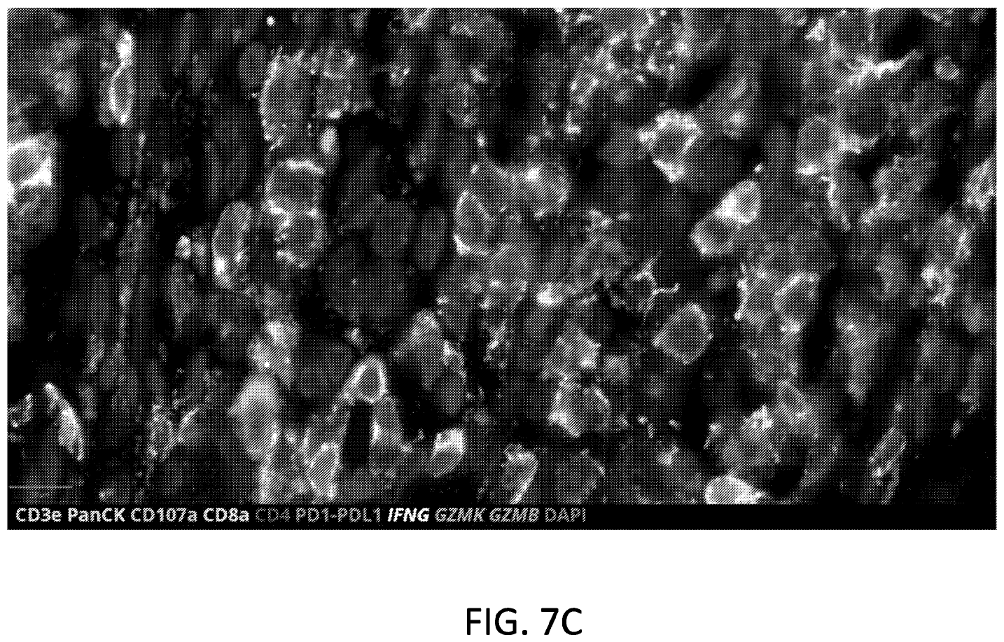

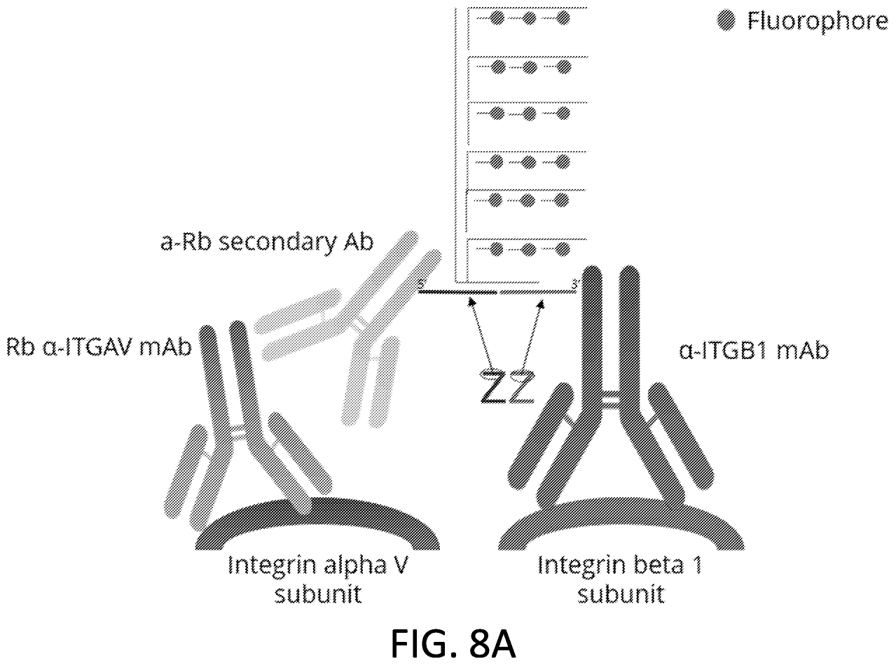

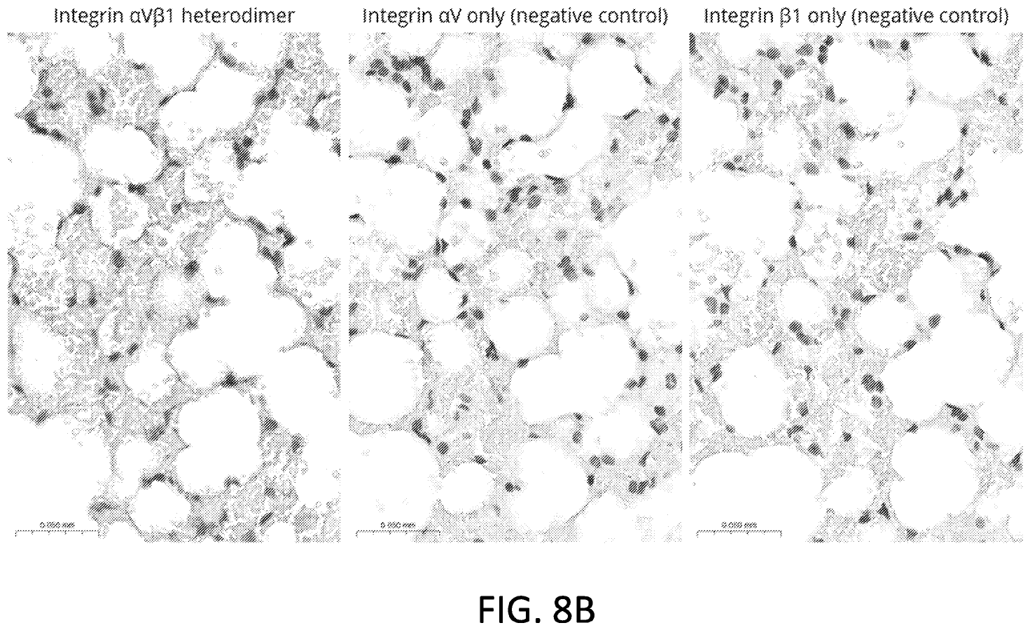

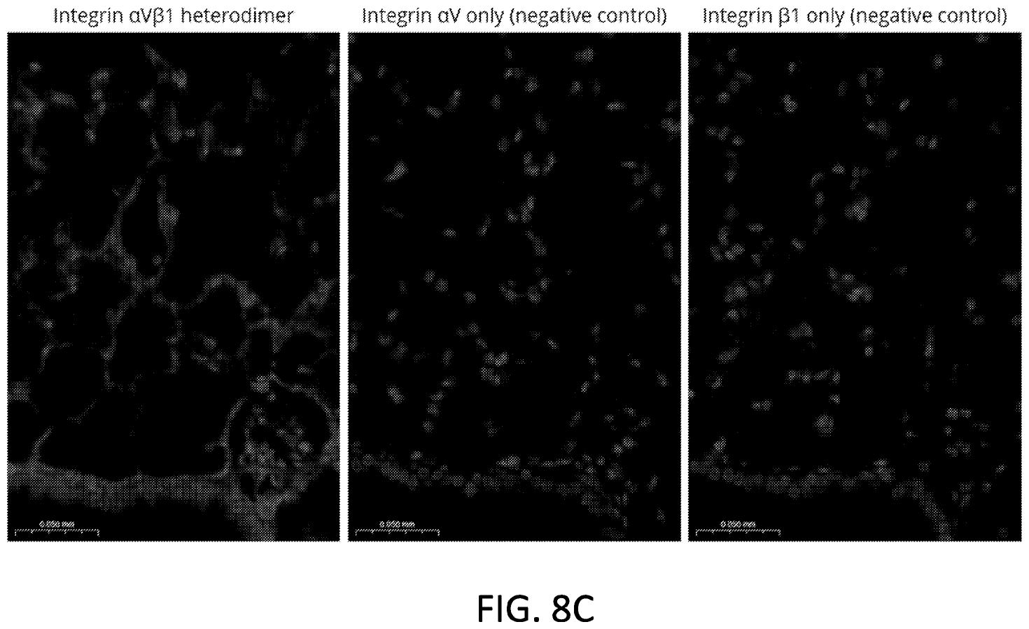

The detection of a single type of protein interaction may be completed with methods for detection of other targets of interest. For example, detection of mRNA can be carried out concomitantly with the detection of the protein interaction. As shown in FIG. 5, detection of PD1/PD-L interactions can be paired with mRNA detection of Hs-IFNγ on lung cancer tissue sample.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method for detecting protein interactions in a biological sample, the method comprising:
(i) contacting a biological sample with a first antibody, or a fragment thereof, covalently attached to a first oligonucleotide;
(ii) contacting the biological sample with a second antibody, or a fragment thereof, covalently attached to a second oligonucleotide;
(iii) contacting the biological sample with a signal-generating complex comprising a nucleic acid component capable of hybridizing to the first and second oligonucleotides; and
(iv) detecting a signal from the signal-generating complex.

2. The method of claim 1, wherein the first antibody and/or the second antibody, or fragment thereof, is selected from a monoclonal antibody, a multispecific antibody, a bispecific antibody, a trispecific antibody, a tetravalent antibody, a single-domain antibody, a chimeric antibody, and a polyclonal antibody mixture.

3. The method of claim 1 or claim 2, wherein the first antibody and/or the second antibody, or fragment thereof, is selected from a Fab, a scFv, a Fv, a scFv-Fc, a Fab′, a Fab′-SH, a F(ab′)₂, a diabody, a minibody, and a tribody.

4. The method of any one of claims 1-3, wherein the first and/or the second oligonucleotide has a length of about 5 to about 100 nucleotides.

5. The method of any one of claims 1-4, wherein the first and/or the second oligonucleotide is covalently attached to the antibody via a linker.

6. The method of any one of claims 1-5, wherein:
step (i) and step (ii) are performed simultaneously;
step (i) is performed before step (ii); or
step (ii) is performed before step (i).

7. The method of any one of claims 1-6, wherein step (i) and/or step (ii) comprises contacting the biological sample with the first and/or second antibody for about 10 minutes to about 48 hours.

8. The method of any one of claims 1-7, wherein step (i) and/or step (ii) comprises contacting the biological sample with the first and/or second antibody at about 4° C. to about 75° C.

9. The method of claim 8, wherein step (i) and/or step (ii) comprises contacting the biological sample with the first and/or second antibody at room temperature.

10. The method of any one of claims 1-9, further comprising contacting the biological sample with a blocking agent before step (i) and/or step (ii).

11. The method of claim 10, wherein the blocking agent comprises a protein, polypeptide, or nucleic acid.

12. The method of any one of claims 1-11, further comprising contacting the biological sample with a crosslinking agent after steps (i) and (ii) but before step (iii).

13. The method of claim 12, wherein the crosslinking agent is a fixative.

14. The method of claim 12 or 13, comprising contacting the biological sample with the crosslinking agent for about 5 minutes to about 24 hours, at a temperature of about 4° C. to about 60° C.

15. The method of any one of claims 12-14, further comprising contacting the biological sample with a protease after the crosslinking agent and before step (iii).

16. The method of any one of claims 1-15, wherein the method further comprises contacting the biological sample with a target probe set comprising a first target probe capable of hybridizing to the first oligonucleotide and to a section of the nucleic acid component of the signal-generating complex, and a second target probe capable of hybridizing to the second oligonucleotide and to a section of the nucleic acid component of the signal-generating complex.

17. The method of claim 16, wherein the first target probe comprises a target (T) section and a label (L) section, wherein the T section comprises a nucleic acid sequence complementary to a section of the first oligonucleotide and the L section comprises a nucleic acid sequence complementary to a section of the nucleic acid component of the second signal-generating complex; and wherein the second target probe comprises a target (T) section and a label (L) section, wherein the T section comprises a nucleic acid sequence complementary to a section of the second oligonucleotide and the L section comprises a nucleic acid sequence complementary to a section of the nucleic acid component of the second signal-generating complex.

18. The method of claim 17, and wherein the L sections are complementary to non-overlapping sections of the nucleic acid component of the second signal-generating complex.

19. The method of claim 17 or 18, wherein the T sections are 3′ of the L sections.

20. The method of claim 17 or 18, wherein the T sections are 5′ of the L sections.

21. The method of any one of claims 17-20, wherein the T sections are at least 10 nucleotides in length, and wherein the L sections are at least 10 nucleotides in length.

22. The method of any one of claims 1-21, wherein the signal-generating complex comprises a pre-pre-amplifier, a pre-amplifier, and/or an amplifier; and one or more label probes, wherein each label probe comprises a detectable label.

23. The method of any one of claims 1-22, wherein the signal-generating complex comprises a pre-amplifier and an amplifier; and one or more label probes, wherein each label probe comprises a detectable label.

24. The method of claim 22 or 23, wherein the detectable label comprises a fluorescent moiety or a chromogenic moiety.

25. The method of any one of claims 22-24, wherein the detectable label comprises a cleavable label.

26. The method of any one of claims 1-25, further comprising:
(iv) contacting the biological sample with one or more third antibody, or a fragment thereof, and one or more fourth antibody, or a fragment thereof, wherein each of the third and fourth antibodies are covalently attached to an oligonucleotide; and
(v) contacting the biological sample with one or more additional signal-generating complex comprising a nucleic acid component capable of hybridizing to the oligonucleotides covalently attached to the third and fourth antibodies.

27. The method of claim 26, wherein step (iv) and step (v) are performed simultaneously.

28. The method of claim 26 or 27, wherein:
step (iv) and/or step (v) is performed before step (ii);
step (iv) and/or step (v) is performed after step (ii); or
step (iv) and/or step (v) is performed after step (iii).

29. The method of any one of claims 1-28, further comprising contacting the biological sample with one of more nucleic acid detection agents.

30. The method of any one of claims 1-29, wherein the biological sample is a tissue specimen or is derived from a tissue specimen.

31. The method of any one of claims 1-29, wherein the biological sample is a blood sample or is derived from a blood sample.

32. The method of any one of claims 1-29, wherein the biological sample is a cytological sample or is derived from a cytological sample.

33. The method of any one of claims 1-29, wherein the biological sample comprises cultured cells.

34. The method of any one of claims 1-33, wherein the first antibody binds directly to an epitope on a first target protein, and wherein the second antibody binds directly to an epitope on a second target protein.

35. The method of any one of claims 1-33, wherein the first antibody binds indirectly to an epitope on a first target protein, and wherein the second antibody binds indirectly to an epitope on a second target protein.

36. The method of any one of claims 1-33, wherein the first antibody binds to an epitope on a first primary antibody that directly binds to an epitope on a first target protein, and wherein the second antibody binds to an epitope on a second primary antibody that directly binds to an epitope on a second target protein.

37. The method of any one of claims 34-36, wherein the first target protein and the second target protein are expressed on the surface of the same cell, and wherein the signal produced from the signal generating complex indicates that the first target protein and the second target protein are in close proximity.

38. The method of any one of claims 34-36, wherein the first target protein and the second target protein are expressed on the surface of different cells, and wherein the signal produced from the signal generating complex indicates that the first target protein and the second target protein are in close proximity.

39. The method of any one of claims 1-33, wherein the first antibody binds directly to a first epitope on a target protein, and wherein the second antibody binds directly to a second epitope on the same target protein.

40. The method of any one of claims 1-33, wherein the first antibody binds indirectly to a first epitope on a target protein, and wherein the second antibody binds indirectly to a second epitope on the same target protein.

41. The method of any one of claims 1-33, wherein the first antibody binds to a first primary antibody that directly binds to a first epitope on a target protein, and wherein the second antibody binds to a second primary antibody that directly binds to a second epitope on the same target protein.

42. The method of any one of claims 39-41, wherein the signal produced from the signal generating complex indicates that the first epitope of the target protein and the second epitope of target protein are in close proximity.

43. A method for detecting protein interactions in a biological sample, the method comprising:
(i) contacting a biological sample with a first antibody, or fragment thereof, covalently attached to a first oligonucleotide, wherein the first antibody binds a first target epitope;
(ii) contacting the biological sample with a second antibody, or fragment thereof, covalently attached to a second oligonucleotide, wherein the second antibody binds a second target epitope;
(iii) contacting the biological sample with a pre-amplifier capable of hybridizing to the first and second oligonucleotides simultaneously, wherein the pre-amplifier comprises binding sites for a plurality of amplifiers;
(iv) contacting the biological sample with the plurality of amplifiers capable of hybridizing to the pre-amplifier, wherein the plurality of amplifiers comprises binding sites for a plurality of label probes;
(v) contacting the biological sample with the plurality of label probes capable of hybridizing to the plurality of amplifiers, wherein each label probe comprises a detectable label; and
(vi) detecting a signal generated from the plurality of label probes when the first target epitope and the second target epitope are in sufficiently close proximity to allow binding of the pre-amplifier to the first and second oligonucleotides simultaneously.

44. The method of claim 43, wherein the method further comprises contacting the biological sample with a target probe set after steps (i) and (ii).

45. The method of claim 44, wherein the target probe set comprises a first target probe capable of hybridizing to the first oligonucleotide and to a section of the pre-amplifier, and a second target probe capable of hybridizing to the second oligonucleotide and to a section of the pre-amplifier.

46. The method of claim 45, wherein the pre-amplifier is capable of hybridizing to the first and second target probes simultaneously.

47. The method of claim 45 or 46, wherein the first target probe comprises a target (T) section and a label (L) section, wherein the T section comprises a nucleic acid sequence complementary to a section of the first oligonucleotide and the L section comprises a nucleic acid sequence complementary to a section of the pre-amplifier; and wherein the second target probe comprises a target (T) section and a label (L) section, wherein the T section comprises a nucleic acid sequence complementary to a section of the second oligonucleotide and the L section comprises a nucleic acid sequence complementary to a section of the pre-amplifier.

48. The method of claim 47, and wherein the L sections are complementary to non-overlapping sections of the nucleic acid component of the pre-amplifier.

49. The method of any one of claims 44-48, wherein the first antibody binds directly to the first epitope, and wherein the second antibody binds directly to the second epitope.

50. The method of any one of claims 44-48, wherein the first antibody binds indirectly to the first epitope, and wherein the second antibody binds indirectly to a second epitope

51. The method of any one of claims 44-48, wherein the first antibody binds to an epitope on a first primary antibody that directly binds to the first epitope, and wherein the second antibody binds to an epitope on a second primary antibody that directly binds to the second epitope.

52. The method of any one of claims 49-51, wherein the first epitope and the second epitope are on the same target protein.

53. The method of any one of claims 49-51, wherein the first epitope is on a first target protein and the second epitope is on a second target protein.

54. The method of claim 53, wherein the first target protein and the second target protein are expressed on the surface of the same cell, and wherein the signal produced from the signal generating complex indicates that the first target protein and the second target protein are in close proximity.

55. The method of claim 53, wherein the first target protein and the second target protein are expressed on the surface of different cells, and wherein the signal produced from the signal generating complex indicates that the first target protein and the second target protein are in close proximity.

56. A kit for detecting protein interactions in a biological sample, comprising:
(i) a first antibody, or a fragment thereof, covalently attached to a first oligonucleotide, and a second antibody, or a fragment thereof, covalently attached to a second oligonucleotide; and
(ii) a signal-generating complex, wherein the signal-generating complex comprises a nucleic acid component capable of hybridizing to the first and/or second oligonucleotide.

57. The kit of claim 56, further comprising:
(iii) one or more third antibody, or a fragment thereof, and one or more fourth antibody, or a fragment thereof, wherein each of the third and fourth antibodies are covalently attached to an oligonucleotide; and
(iv) one or more additional signal-generating complex wherein the signal-generating complex comprises a nucleic acid component capable of hybridizing to the oligonucleotides covalently attached to the third and fourth antibodies.

58. The kit of claim 56 or 57, wherein the antibody or fragment thereof is selected from a monoclonal antibody, a multispecific antibody, a bispecific antibody, a trispecific antibody, a tetravalent antibody, a single-domain antibody, a chimeric antibody, and a polyclonal antibody mixture.

59. The kit of any of claims 56-58, wherein the antibody or fragment thereof is selected from a Fab, a scFv, a Fv, a scFv-Fc, a Fab′, a Fab′-SH, a F(ab′)₂, a diabody, a minibody, and a tribody.

60. The kit of any one of claims 56-59, wherein the oligonucleotide has a length of about 10 to about 100 nucleotides.

61. The kit of any one of claims 56-60, wherein the oligonucleotide is covalently attached to the antibody via a linker.

62. The kit of any one of claims 56-61, wherein the signal-generating complex comprises a pre-pre-amplifier, a pre-amplifier, and/or an amplifier; and one or more label probes, wherein each label probe comprises a detectable label.

63. The kit of any one of claims 56-62, wherein the signal-generating complex comprises a pre-amplifier and an amplifier; and one or more label probes, wherein each label probe comprises a detectable label.

64. The kit of claim 62 or 63, wherein the detectable label comprises a fluorescent moiety or a chromogenic moiety.

65. The kit of any one of claims 56-64, further comprising a blocking agent, a crosslinking agent, a protease, or any combination thereof.

66. The kit of any one of claims 56-65, further comprising instructions for carrying out a method of detecting the protein interactions in the biological sample.

67. The kit of any one of claims 56-66, further comprising a target probe set, wherein the target probe set comprises a first target probe capable of hybridizing to the first oligonucleotide and to a section of the nucleic acid component of the signal-generating complex, and a second target probe capable of hybridizing to the second oligonucleotide and to a section of the nucleic acid component of the signal-generating complex.

68. The kit of claim 67, wherein the first target probe comprises a target (T) section and a label (L) section, wherein the T section comprises a nucleic acid sequence complementary to a section of the first oligonucleotide and the L section comprises a nucleic acid sequence complementary to a section of the nucleic acid component of the signal-generating complex; and wherein the second target probe comprises a target (T) section and a label (L) section, wherein the T section comprises a nucleic acid sequence complementary to a section of the second oligonucleotide and the L section comprises a nucleic acid sequence complementary to a section of the nucleic acid component of the signal-generating complex.

69. The kit of claim 68, and wherein the L sections are complementary to non-overlapping sections of the nucleic acid component of the second signal-generating complex.
